(19) United States
(12) Patent Application Publication
(10) Pub. No.: US 2025/0017833 A1
(43) Pub. Date: Jan. 16, 2025

(54) MALODOR-COUNTERACTING COMPOUNDS, COMPOSITIONS, AND USES THEREOF

(71) Applicant: INTERNATIONAL FLAVORS AND FRAGRANCES INC., NEW YORK, NY (US)

(72) Inventors: Allyson Battaglia, Union Beach, NJ (US); Christophe Finel, Union Beach, NJ (US); Marie Demazure, Union Beach, NJ (US); Evan Beach, Fanwood, NJ (US); Clémentine Marteau-Roussy, Union Beach, NJ (US)

(21) Appl. No.: 18/702,050

(22) PCT Filed: Oct. 19, 2022
(86) PCT No.: PCT/US2022/047170

§ 371 Date: Apr. 17, 2024

Related U.S. Application Data
(60) Provisional application No. 63/257,683, filed on Oct. 20, 2021.

(51) Int. Cl. A61K 8/41; A61L 9/012; A61L 9/014; A61L 101/32; A61L 101/34; A61L 101/36; A61L 101/44; A61Q 15/00; C11D 3/30; C11D 3/50

(52) U.S. Cl. CPC A61K 8/41; A61L 9/012; A61L 9/014; A61Q 15/00; C11D 3/30; C11D 3/505; A61L 2101/32; A61L 2101/34; A61L 2101/36; A61L 2101/44

(57) ABSTRACT

Provided herein are compounds, compositions, and methods for counteracting malodor and providing longevity and durability to fragrance ingredients. Also provided are consumer products including malodor-counteracting compounds or compositions.

### CROSS-REFERENCE TO RELATED APPLICATION

This application is a 371 if International Application No. PCT/US2022/47170, filed Oct. 19, 2022, and claims priority from U.S. Provisional Application No. 63/257,683, filed Oct. 20, 2021, the contents of which are hereby incorporated by reference in their entirety.

### FIELD OF THE INVENTION

Provided herein are compounds, compositions, and methods for counteracting malodor and providing longevity and durability to fragrance ingredients. Also provided are consumer products including malodor-counteracting compounds or compositions.

### BACKGROUND

Numerous methods have been developed to prevent or reduce body and environmental malodors. For example, conventional perfumes including a variety of fragrance materials have been developed to mask malodors, which generally function via two mechanisms: first, the fragrance materials blend with the malodor compound to provide a different and more desirable aroma; and second, the fragrance materials are employed in a large quantity to overwhelm the malodor compound. In some cases, the perfume may include olfactory receptor blockers to inhibit the detection of malodor. However, none of these methods directly treat the source of the malodor. Thus, there remains a need in the art for compositions that address the source of malodor.

### SUMMARY OF THE INVENTION

In an aspect is provided a malodor-counteracting composition including: (a) a 2,2′(alkylimino)bis[ethanol], wherein the alkyl is a C₄₋₁₂ alkyl; and (b) one or more fragrance ingredients susceptible to hydrolysis, ethanolysis, solvolysis, retro-aldol elimination, dimerization, polymerization, and/or transesterification.

In an aspect is provided a malodor-counteracting composition including a 2,2′-(alkylimino)bis[ethanol], wherein the alkyl is a C₄₋₁₂ alkyl, and one or more of: (i) a solvent selected from the group consisting of IPM, DPG, triethyl citrate, and any combination thereof; (ii) an antibacterial active selected from the group consisting of an antibacterial alcohol, a bactericidal acid, a diol, a polyol, a quaternary ammonium compound, silver metal, silver salt, and any combination thereof; (iii) a water absorber selected from the group consisting of clay, aluminum salt, magnesium oxide, talc, polyacrylate, cellulose, magnesium sulfate, and any combination thereof; (iv) a stabilizer selected from the group consisting of a UV filter, an antioxidant, a chelating agent, and any combination thereof; or (v) an olfactory receptor blocker.

In an aspect is provide a consumer product comprising a malodor-counteracting composition described herein.

In an aspect is provided a method of counteracting a malodor in an air space or a substrate, including introducing into the air space or the substrate a composition described herein or a consumer product described herein.

Each of the aspects and embodiments described herein are capable of being used together, unless excluded either explicitly or clearly from the context of the embodiment or aspect.

### BRIEF DESCRIPTION OF THE DRAWINGS

FIG. 1 shows sensory panel intensity ratings (mean±SD; 0—lowest to 10—highest) of sweat malodor in fabric samples treated with an unfragranced fabric refresher spray, the same fabric refresher spray containing fragrance (0.1% Floral HCA), or the fabric refresher spray containing 0.5% of exemplary malodor-counteracting compound 2,2′-(octylimino)bis[ethanol] (0.5% C8).

FIG. 2 shows the mean and standard deviation of sweat malodor intensity ratings (0—lowest to 10—highest) reported by subjects of their axilla before (T8 h) and after (T12 h) a workout session. The “wash-out phase” portion of the graph shows sweat malodor intensity ratings of axilla when subjects used an unfragranced shower gel and no underarm product, which served as a baseline. The “test phase” portion of the graph shows sweat malodor intensity rating of axilla when subjects used the unfragranced shower gel and two underarm products (one to each axilla) with one underarm product being a non-antiperspirant spray (Non-AP Spray) and the other the same Non-AP spray including 0.5% of exemplary malodor-counteracting compound 2,2′-(octylimino)bis[ethanol] (Non-AP Spray+C8 @0.5%).

### DETAILED DESCRIPTION OF THE INVENTION

“Malodor” is a term used to describe undesirable or unpleasant odor. Common sources of malodors include, but are not limited to, the human body, e.g., perspiration/sweat odor, foot odor, bad breath (e.g., halitosis), axillary odor, scalp odor, and aging odor, and environmental odors such as smoke (e.g., cigarette smoke, cigar smoke), mold, mildew, bathroom odors (e.g., excrement, urine), pet odors, and kitchen waste. Strategies for controlling body and environmental malodors have typically focused on eliminating the perception of malodor by overwhelming the malodor with high concentrations of fragrance ingredients, applying fragrance ingredients that mix with the malodor compounds to produce a more pleasant odor, and/or using olfactory receptor blockers to prevent sensory detection of the malodor. However, these strategies come with certain drawbacks. For example, the use of high concentrations of fragrance ingredients can in itself result in an unpleasant odor, for example due to the intensity of the fragrance. Furthermore, these strategies do not counteract the malodor compounds themselves, which suggests that the malodor remains and may be detectable if the strategy (e.g., fragrance, receptor blocker) is not sufficiently long-lasting. There is thus a need for compositions and methods for counteracting, not just masking, malodor.

As described herein (see, e.g., Examples), it was surprisingly found that 2,2′-(alkylimino)bis[ethanol]s are effective in counteracting malodors, without the need to use fragrances or olfactory receptor blockers. Thus, provided herein are compositions and methods that include the use of 2,2′-(alkylimino)bis[ethanol] to counteract malodor. 2,2′-(alkylimino)bis[ethanol]s may be referred to herein as malodor-counteracting compounds.

In some cases, it may be desirable to have a composition that includes malodor-counteracting compounds and fragrance ingredients. In this way, the composition may both counteract the malodor itself and impart a desirable scent. An advantage of a composition with malodor counteracting abilities and fragrance is that the concentration of fragrance ingredients required will be less than what would be needed to overwhelm the malodor since the malodor itself will be reduced or prevented. This is advantageous from a cost-savings perspective and in the ability to prevent introducing a fragrance that is undesirable due to its intensity.

However, the ability to combine malodor-counteracting compounds with a fragrance can be challenging because fragrances typically include natural and synthetic ingredients capable of undergoing a range of chemical reactions. By way of example, aldehyde and ester functional groups are ubiquitous in fragrance formulations, and are used frequently in top, middle, and base notes. Aldehydes may degrade by various mechanisms such as oxidation or dimerization. Esters are vulnerable to cleavage or transesterification with alcohol-functional molecules such as solvents (hydrolysis, ethanolysis, etc.) or other formulation components. These degradation processes may also be intensified in the presence of catalysts.

A 2,2′-(alkylimino)bis[ethanol] having an alkyl group in the range of C₁₋₂₀ may be expected to create stability issues with aldehydes and esters because the amine functional group could act as a chemical base, potentially promoting a base-catalyzed degradation process. The primary alcohols found in the bis[ethanol] portion could potentially attack esters, causing transfer of the carbonyl group from the fragrance ingredient to the 2,2′-(alkylimino)bis[ethanol]. The overall effect of these processes would be to change the chemical composition of the fragrance, which could harm the aesthetic effects or other benefits of the composition, such as long-lastingness, freshness, and/or malodor coverage.

In considering 2,2′-(alkylimino)bis[ethanol]s having an alkyl group in the range of C₁₋₂₀, the lower chain length analogs have a higher amine content and primary hydroxyl group content, per unit mass, and therefore could be expected to increase degradation when compared at equal mass loading. However, it was surprisingly found that the magnitude of degradation varied depending on the fragrance ingredient, with some ingredients insensitive to the alkyl chain length of the amine and others showing clear differences for example in the range of C₁₋₁₂. It was unexpectedly found that 2,2′-(alkylimino)bis[ethanol] having an alkyl group in the range of C₄₋₁₂ could be combined with certain fragrance ingredients without detrimental effects.

Thus, in aspects are provided compositions containing 2,2′-(alkylimino)bis[ethanol] having an alkyl group in the range of C₄₋₁₂ that counteract malodor and fragrance ingredients that are insensitive or have reduced sensitivity to degradation by 2,2′-(alkylimino)bis[ethanol] having an alkyl group in the range of C₄₋₁₂. In some embodiments, the fragrance ingredients include one or more of an aldehyde, an aldehyde precursor, an ester, an ester precursor, a lactone, or a lactone precursor. A precursor as used herein refers to a compound that once formulated into an accord, full fragrance, or consumer product (interchangeably referred to herein also as a functional product) will convert to an aldehyde, an ester, or a lactone once formulated into an accord, full fragrance, or consumer product as a result of a chemical reaction. In some embodiments, the compositions and consumer products containing 2,2′-(alkylimino)bis[ethanol] having an alkyl group in the range of C₄₋₁₂ and the one or more fragrance ingredients exhibit increased fragrance retention and longer lasting fragrance perception.

In some embodiments, when malodor-counteracting compounds (see, e.g., Section I-A) or malodor-counteracting compositions (see, e.g., Section I-B) are present in consumer products containing a fragrance, such as personal care products (see, e.g., Section I-C), the products possess improved fragrance retention, longer lasting fragrance perception, and a malodor counteracting effect.

The compounds, compositions, consumer products, and methods provided herein are advantageous in their ability to directly counteract malodor without the need to use strong fragrances or olfactory receptor blockers, although the use of fragrances and receptor blockers is not precluded. Indeed, combining malodor-counteracting compounds described herein with fragrance and/or receptor blockers may enhance the malodor coverage effect. The compounds, compositions, consumer products, and methods are advantageous in that they may further include fragrance ingredients that are insensitive or have reduced sensitivity to the malodor-counteracting compound. It is further advantageous that the fragrance ingredients need not be present in the compositions provided herein at high levels to mask malodor, which may offer beneficial health and/or environmental effects, e.g., reduced chances of allergic reaction or irritation, and cost-savings. The compounds, compositions, consumer products, and methods provided herein thus offer an effective, efficient, environmentally friendly, and economic means of counteracting malodor.

Unless defined otherwise, all technical and scientific terms used herein have the same meaning as commonly understood by one of ordinary skill in the art to which this disclosure belongs.

This disclosure is not limited by the exemplary methods and materials disclosed herein, and any methods and materials similar or equivalent to those described herein can be used in the practice or testing of embodiments of this disclosure.

The headings provided herein are not limitations of the various aspects or embodiments of this disclosure which can be had by reference to the specification as a whole. The section headings used herein are for organizational purposes only and are not to be construed as limiting the subject matter described. Any terms defined are more fully defined by reference to the specification as a whole.

All publications, including patent documents, scientific articles, and databases, referred to in this application are incorporated by reference in their entirety for all purposes to the same extent as if each individual publication were individually incorporated by reference. Nothing herein is to be construed as an admission that such publications constitute prior art. If a definition set forth herein is contrary to or otherwise inconsistent with a definition set forth in the patents, applications, published applications and other publications that are herein incorporated by reference, the definition set forth herein prevails over the definition that is incorporated herein by reference.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### Definitions

Definitions of terms may appear throughout the specification. It is to be understood that this disclosure is not limited to particular embodiments described, as such may, of course, vary. It is also to be understood that the terminology used herein is for the purpose of describing particular embodiments only and is not intended to be limiting.

It must be noted that as used herein and in the appended claims, the singular forms “a”, “an”, and “the” include plural referents unless the context clearly dictates otherwise. For example, “a” or “an” include “at least one” and “one or more.”

The terms “comprising”, “comprises” and “comprised of” as used herein are synonymous with “including”, “includes”, “containing”, “contains”, and grammatical variants thereof, are inclusive or open-ended and do not exclude additional, non-recited members, elements, or method steps. The terms “comprising”, “comprises” and “comprised of”, “including”, “includes” or “containing”, “contains”, and grammatical variants thereof also include the term “consisting of”.

The abbreviations used herein have their conventional meaning within the chemical and biological arts. As used herein all percentages are weight percent (% wt) unless otherwise noted, mmHg is understood to be millimeters of mercury, M is understood to be moles per Liter, ppm is understood to stand for parts per million, L is understood to be liter, mL is understood to be milliliter, Kg is understood to be kilogram, g be gram, min is understood to be minute, and h is understood to be hour.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

Values and ranges may be presented herein with numerical values being preceded by the term “about.” The term “about” is used herein to provide literal support for the exact number that it precedes as well as a number that is near to or approximately the number that the term precedes. In determining whether a number is near to or approximately a specifically recited number, the near or approximating unrecited number can be a number which, in the context in which it is presented, provides the substantial equivalent of the specifically recited number. For example, in connection with a numerical value, the term “about” refers to a range of −10% to +10% of the numerical value, unless the term is otherwise specifically defined in context. All values and ranges may implicitly include the term “about” unless the context dictates otherwise.

As used herein, the term “consumer” means both the user of the fragrance composition and the observer nearby or around the user.

The terms “fragrance,” “fragrance composition,” “fragrance formulation,” and “perfume composition,” including grammatical variants thereof, mean the same and refer to a composition that is a mixture of fragrance ingredients including, for example, alcohols, aldehydes, terpenes, ketones, esters, ethers, lactones, nitriles, natural oils, synthetic oils, mercaptans, etc., which are admixed so that the combined odors of the individual ingredients produce a fragrance. In some embodiments, the fragrance is an accord. In some embodiments, the fragrance is a full fragrance. Fragrance ingredients include, but are not limited to essential oils, natural extracts, and synthetic ingredients.

Where substituent groups are specified by their conventional chemical formulae, written from left to right, they equally encompass the chemically identical substituents that would result from writing the structure from right to left, e.g., —CH₂O— is equivalent to —OCH₂—.

Certain chemical functional groups named here are preceded by a shorthand notation indicating the total number of carbon atoms that are to be found in the indicated chemical group. For example: C₁₋₂₀ alkyl describes an alkyl group having a total of 1 to 20 carbon atoms (e.g. C₁₀ implies C₁₀H₂₁). The total number of carbons in the shorthand notation does not include carbons that may exist in substituents of the group described. Unless specified to the contrary, the following terms have the following meaning:
“Azido” refers to the —N₃ functional group.
“Cyano” refers to the —CN functional group.
“Halo” refers to fluoro, chloro, bromo, or iodo.
“Halide” refers to a halide atom bearing a negative charge such as for example, fluoride (F⁻), chloride (Cl⁻), bromide (Br⁻), or iodide (I⁻).
“Hydroxyl” refers to the —OH functional group.
“Nitro” refers to the —NO₂ functional group.
“Oxo” refers to the =O substituent.
“Amide” used herein refers to a group represented by:

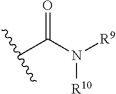

wherein R⁹ and R¹⁰ each independently represent a hydrogen or hydrocarbyl group, or R⁹ and R¹⁰ taken together with the N atom to which they are attached complete a hetero cycle having from 4 to 8 atoms in the ring structure.

“Amine” and “amino” are art recognized terms and refer to both unsubstituted and substituted amines and salts thereof, e.g., a moiety that can be represented by:

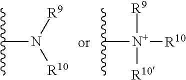

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₁₋₁₂, C₁₋₁₄, C₁₋₁₆, C₁₋₁₈, C₁₋₂₀, C₂₋₃, C₂₋₄, C₂₋₅, C₂₋₆, C₂₋₇, C₂₋₈, C₂₋₉, C₂₋₁₀, C₂₋₁₂, C₂₋₁₄, C₂₋₁₆, C₂₋₁₈, C₂₋₂₀ C₃₋₄, C₃₋₅, C₃₋₆, C₃₋₇, C₃₋₈, C₃₋₉, C₃₋₁₀, C₃₋₁₂, C₃₋₁₄, C₃₋₁₆, C₃₋₁₈, C₃₋₂₀, C₄₋₅, C₄₋₆, C₄₋₇, C₄₋₈, C₄₋₉, C₄₋₁₀, C₄₋₁₂, C₄₋₁₄, C₄₋₁₆, C₄₋₁₈, C₄₋₂₀, C₅₋₆, C₅₋₇, C₅₋₈, C₅₋₉, C₅₋₁₀, C₅₋₁₂, C₅₋₁₄, C₅₋₁₆, C₅₋₁₈, and C₅₋₂₀. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂-C₃, C₂-C₄, C₂-C₅, C₂-C₆, C₂-C₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆.

Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkenylene” refers to an alkenyl group, as defined above, linking at least two other groups, i.e., a divalent hydrocarbon radical. The two moieties linked to the alkenylene can be linked to the same atom or different atoms of the alkenylene. Alkenylene groups include, but are not limited to, ethenylene, propenylene, isopropenylene, butenylene, isobutenylene, sec-butenylene, pentenylene and hexenylene. Alkenylen groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, isobutynyl, scc-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkynylene” refers to an alkynyl group, as defined above, linking at least two other groups, i.e., a divalent hydrocarbon radical. The two moieties linked to the alkynylene can be linked to the same atom or different atoms of the alkynylene. Alkynylene groups include, but are not limited to, ethynylene, propynylene, isopropynylene, butynylene, sec-butynylene, pentynylene and hexynylene. Alkynylene groups can be substituted or unsubstituted.

“Alkylhydroxy” or “hydroxyalkyl” refers to an alkyl group, as defined above, where at least one of the hydrogen atoms is replaced with a hydroxy group. As for the alkyl group, alkylhydroxy groups can have any suitable number of carbon atoms, such as C₁₋₆. Exemplary alkylhydroxy groups include, but are not limited to, hydroxy-methyl, hydroxyethyl (where the hydroxy is in the 1- or 2-position), hydroxypropyl (where the hydroxy is in the 1-, 2- or 3-position), hydroxybutyl (where the hydroxy is in the 1-, 2-, 3- or 4-position), hydroxypentyl (where the hydroxy is in the 1-, 2-, 3-, 4- or 5-position), hydroxyhexyl (where the hydroxy is in the 1-, 2-, 3-, 4-, 5- or 6-position), 1,2-dihydroxyethyl, and the like.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Halogen” refers to fluorine, chlorine, bromine, and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluorocthoxy, perfluoroethoxy, etc.

“Cycloalkyl” refers to a saturated or partially unsaturated, monocyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, _{C3-10}, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbomane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbomene, and norbomadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Cycloalkylene” refers to a cycloalkyl group having the number of carbon atoms indicated, and linking at least two other groups, i.e., a divalent radical. The two moieties linked to the cycloalkylene can be linked to the same atom or different atoms of the cycloalkylene group. Examples of cycloalkylene rings include cyclopropylene, cyclobutylene, cyclopentylene and cyclohexylene, among others. Cycloalkylene groups can be linked 1,1, 1,2, 1,3, or 1,4. The cyclohexylene ring, for example, can adopt a number of conformations, including the boat and chair conformations. The chair conformation of cyclohexylene can have substituents in an axial or equatorial orientation. The divalent nature of the cycloalkylenes results in cis and trans formations where cis refers to both substituents being on the same side (top or bottom) of the cycloalkylene ring, and where trans refers to the substituents being on opposite sides of the cycloalkylene ring. For example, cis-1,2- and cis-1,4-cyclohexylene can have one substituent in the axial orientation and the other substituent in the equatorial orientation, while trans-1,2- and trans-1,4-cyclohexylene have both substituents in the axial or equatorial orientation. cis-1,3-cyclohexylene have both substituents in the axial or equatorial orientation, and trans-1,3-cyclohexylene can have one substituent in the axial orientation and the other substituent in the equatorial orientation. Cycloalkylene groups can be substituted or unsubstituted.

“Heterocycloalkyl” refers to a saturated ring system having from 3 to 12 ring members and from 1 to 4 heteroatoms of N, O and S. Additional heteroatoms can also be useful, including, but not limited to, B, Al, Si and P. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thictane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl includes 3 to 8 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also form a ring having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Heterocyclalkylene” refers to a heterocyclalkyl group, as defined above, linking at least two other groups. The two moieties linked to the heterocyclalkylene can be linked to the same atom or different atoms of the heterocyclalkylene. Heterocycloalkylene groups can be substituted or unsubstituted.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Arylene” refers to an aryl group, as defined above, linking at least two other groups. The two moieties linked to the aryl can be linked to the same atom or different atoms of the aryl. Arylene groups can be substituted or unsubstituted.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Additional heteroatoms can also be useful, including, but not limited to, B, Al, Si and P. The heteroatoms can also be oxidized, such as, but not limited to, —S(O)— and —S(O)₂—. Heteroaryl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

Some heteroaryl groups include from 5 to 10 ring members and only nitrogen heteroatoms, such as pyrrole, pyridine, imidazole, pyrazole, triazole, pyrazine, pyrimidine, pyridazine, triazine (1,2,3-, 1,2,4- and 1,3,5-isomers), indole, isoindole, quinoline, isoquinoline, quinoxaline, quinazoline, phthalazine, and cinnoline. Other heteroaryl groups include from 5 to 10 ring members and only oxygen heteroatoms, such as furan and benzofuran. Some other heteroaryl groups include from 5 to 10 ring members and only sulfur heteroatoms, such as thiophene and benzothiophene. Still other heteroaryl groups include from 5 to 10 ring members and at least two heteroatoms, such as imidazole, pyrazole, triazole, pyrazine, pyrimidine, pyridazine, triazine (1,2,3-, 1,2,4- and 1,3,5-isomers), thiazole, isothiazole, oxazole, isoxazole, quinoxaline, quinazoline, phthalazine, and cinnoline.

“Heteroarylene” refers to a heteroaryl group, as defined above, linking at least two other groups. The two moicties linked to the heteroaryl are linked to different atoms of the heteroaryl. Heteroarylene groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

The groups defined above and herein may optionally be substituted by any suitable number and type of substituents. Representative substituents include, but are not limited to, halogen, haloalkyl, haloalkoxy, —OR′, ═O, —OC(O)R′, —(O)R′, —O₂R′, —ONR′R″, —OC(O)NR′R″, ═NR′, ═N—OR′, —NR′R″, —NR″C(O)R′, —NR′—(O)NR″R″′, —NR″C(O)OR′, —NH—(NH₂)═NH, —NR′C(NH₂)═NH, —NH—(NH₂)═NR′, —SR′, —S(O)R′, —S(O)₂R′, —S(O)₂NR′R″, —NR′S(O)₂R″, —N₃ and —NO₂, R′, R″ and R′″ each independently refer to hydrogen, unsubstituted alkyl, such as unsubstituted C₁₋₆ alkyl. Alternatively, R′and R″, or R″ and R″′, when attached to the same nitrogen, are combined with the nitrogen to which they are attached to form a heterocycloalkyl or heteroaryl ring, as defined above.

### I. MALODOR-COUNTERACTING COMPOUNDS, COMPOSITIONS, AND CONSUMER PRODUCTS

Provided herein are 2,2′-(alkylimino)bis[ethanol] compounds, where the alkyl is a C₄₋₁₂ alkyl, having malodor counteracting abilities (see, Section I-A, below) and compositions including said compounds (see, Section I-B below). The compounds and/or compositions described herein may be included in a consumer product. The compounds, compositions, and consumer products described herein are useful for counteracting malodor, such as body and/or environmental malodor.

### A. Malodor-Counteracting Compounds

In an aspect is provided a 2,2′-(alkylimino)bis[ethanol], wherein the alkyl is a C₄₋₁₂ alkyl, for counteracting malodor. Such compounds are referred to herein alternatively as malodor-counteracting compounds. In some embodiments, the alkyl of the 2,2′-(alkylimino)bis[ethanol] is a C₄₋₁₀ alkyl. In some embodiments, the alkyl of the 2,2′-(alkylimino)bis[ethanol] is a C₆₋₁₀ alkyl. In some embodiments, the alkyl of the 2,2′-(alkylimino)bis[ethanol] is a C₈ alkyl. In some embodiments, the alkyl is a linear alkyl. In some embodiments, the alkyl of the 2,2′-(alkylimino)bis[ethanol] is a C₈ linear alkyl. A 2,2′-(alkylimino)bis[ethanol] including a C₈ linear alkyl may be referred to alternatively as 2,2′-(octylimino)bis[ethanol].

In some embodiments, the 2,2′-(alkylimino)bis[ethanol] is protonated. In some embodiments, the 2,2′-(alkylimino)bis[ethanol] is in a neutral form. For example, the 2,2′-(alkylimino)bis[ethanol] may be combined with another compound that results in the combination having a neutral pH. In some cases, the neutral combination of 2,2′-(alkylimino)bis[ethanol] and the other compound may be formulated into a composition, e.g., such as compositions described in Section I-B, or a consumer product, e.g., such as consumer products described in Section I-C, to produce a neutral pH composition and/or consumer product.

In some embodiments, the 2,2′-(alkylimino)bis[ethanol] is combined with an acid and/or a salt. In some embodiments, the 2,2′-(alkylimino)bis[ethanol] combined with an acid and/or a salt produces a combination with neutral pH. In some embodiments, the 2,2′-(alkylimino)bis[ethanol] is combined with an acid. Any type of acid capable of producing a pH neutral combination when combined with 2,2′-(alkylimino)bis[ethanol] is contemplated for use herein. In some embodiments, the acid is an organic acid. In some embodiments, the acid is an inorganic acid. In some embodiments, the acid is a plant-derived fatty acid. Non-limiting examples of suitable organic acids include monocarboxylic acids such as short-chain carboxylic acids (e.g., formic acid, acetic acid, propionic acid, butyric acid, valeric acid, hexanoic acid (straight or branched)), fatty acids (e.g., C₁₂ acid, C₁₄ acid, C₁₆ acid, or C₁₈ fatty acid, oleic acid, linoleic acid, linolenic acid and mixtures of thereof, ricinoleic acid), hydroxy acids (e.g., glycolic acid, lactic acid), unsaturated acids (e.g., undecylenic acid), isomerized acids (e.g., butyloctanoic acid, isostearic acid), ascorbic acids, alkylascorbic acids, dehydroacetic acid, benzoic acid, hydroxybenzoic acids (e.g., salicylic acid), resin derivative acids (e.g., abictic acid), PEG ether/carboxylic acids (e.g., Laureth-X carboxylic acids), fragrance acids (e.g., cinnamic acid, p-anisic acid, phenoxyacetic acid, phenylacetic acid, vanillic acid), gluconic acid, pantothenic acid; 2-3+ carboxylic acids such as dioic acids (e.g., oxalic acid, malonic acid, succinic acid, malic acid, tartaric acid, galactaric acid, glutaric acid, levulinic acid, adipic acid, azelaic acid, sebacic acid, C36 dimer acids), unsaturated acids (maleic acid, fumaric acid, itaconic acid), citric acid, 1,2,3-propanetricarboxylic acid, chelators (e.g., ethylenediamine tetraacetic acid (EDTA), pentetic acid), isophthalic acid, phthalic acid; polyacrylic acid; polyglutamic acid; polylactic acid; acrylic acid copolymers; methacrylic acid copolymers; amino acids such as aspartic acid, glutamic acid, amino acid derivatives (e.g., lauryl aspartic acid), uric acid; and sulfonic acids such as methanesulfonic acid, camphorsulfonic acid, toluenesulfonic acid. Non-limiting examples of suitable inorganic acids include boron acids (e.g., boric acid), halogen acids (e.g., hydrochloric acid), phosphorus acids (e.g., phosphoric acid, pyrophosphoric acid) and sulfur acids (e.g., sulfuric acid). Non-limiting examples of suitable plant-derived fatty acids include acids derived from coconut, palm, soy, etc. In some embodiments, the acid is formic acid. In some embodiments, the acid is a C₁₂ fatty acid, C₁₄ fatty acid, C₁₆ fatty acid, C₁₈ fatty acid, oleic acid, linoleic acid, linolenic acid and mixtures of thereof, ricinoleic acid. In some embodiments, the acid is lactic acid. In some embodiments, the acid is undecylenic acid. In some embodiments, the acid is butyloctanoic acid. In some embodiments, the acid is isostearic acid. In some embodiments, the acid is ascorbic acid. In some embodiments, the acid is benzoic acid. In some embodiments, the acid is any one or more of cinnamic acid, p-anisic acid, phenoxyacetic acid, phenylacetic acid, or vanillic acid. In some embodiments, the acid is gluconic acid. In some embodiments, the acid is one or more of oxalic acid, malonic acid, succinic acid, malic acid, tartaric acid, galactaric acid, glutaric acid, levulinic acid, adipic acid, azelaic acid, sebacic acid, or C36 dimer acids. In some embodiments, the acid is one or more of maleic, fumaric, itaconic. In some embodiments, the acid is citric acid. In some embodiments, the acid is 1,2,3-propanetricarboxylic acid. In some embodiments, the acid is EDTA. In some embodiments, the acid is pentetic acid. In some embodiments, the acid is isophthalic acid or phthalic acid. In some embodiments, the acid is aspartic acid or glutamic acid. In some embodiments, the acid is boric acid or hydrochloric acid. In some embodiments, the acid is hydrochloric acid.

In some embodiments, the 2,2′-(alkylimino)bis[ethanol] is combined with a salt. In some embodiments, the salt is HCl. In some embodiments, the 2,2′-(alkylimino)bis[ethanol] is combined with an acid and a salt described herein.

In some embodiments, the malodor-counteracting compound is contained in a capsule. Thus, in some embodiments, the malodor-counteracting compound is encapsulated. In some embodiments, the malodor-counteracting compound in combination with another compound as described herein (e.g., an acid and/or salt), is contained in a capsule, i.e., it is encapsulated.

Capsules, e.g., microcapsules, may be used to deliver, apply, or release the malodor-counteracting compound to a target area in a time-delayed or controlled manner. In some embodiments, the capsules are sustainable capsules. In some embodiments, the capsules are microcapsules. In some embodiments, the capsule includes a polymer. For example, the capsule wall is formed by a polymer. In some embodiments, the polymer is polyacrylate, polyurca, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde) or a combination thereof. Non-limiting examples of capsules, methods of making capsules, and methods of encapsulating ingredients are described in published applications WO2020/131890, WO2020/131866, WO2018053356, US2019/0076811, US2022/0226208, WO2018/006089, WO2019/227019, WO2020/131956, WO2020/131875, WO2020/131879, WO2015/070228, and WO2017/192648, which are incorporated herein by reference in their entirety.

In some embodiments, the 2,2′-(alkylimino)bis[ethanol] described herein counteracts a body malodor. In some embodiments, body malodor is a sweat malodor, foot odor, an axillary odor, scalp odor, and/or an aging odor. In some embodiments, the body malodor is a sweat malodor.

In some embodiments, the 2,2′-(alkylimino)bis[ethanol] described herein counteracts an environmental malodor. In some embodiments, environmental malodor is smoke (e.g., cigarette, cigar smoke), mold, mildew, bathroom odors (e.g., excrement, urine), pet odors, and/or kitchen waste malodor. In some embodiments, the environmental malodor is bathroom malodor. In some embodiments, the environmental malodor is mildew. In some embodiments, the environmental malodor is mold. In some embodiments, the environmental malodor is smoke.

In some embodiments, the 2,2′-(alkylimino)bis[ethanol] described herein counteracts a malodor described herein that is present in an air space. Non-limiting examples of air spaces containing malodor to be counteracted include homes, offices, gyms, etc., and rooms therein, e.g., bathrooms, kitchens, bedrooms, living rooms, locker rooms, workout rooms, garages, etc.

In some embodiments, the 2,2′-(alkylimino)bis[ethanol] counteracts a malodor described herein that is present on a substrate. In some embodiments, the substrate is a fabric. Non-limiting examples of fabric include clothing, furniture, curtains, drapes, wall hangings, carpets, rugs, etc. In some embodiments, the substrate is human tissue. In some embodiments, the human tissue is axillary, scalp, face, or foot skin.

As described below, the 2,2′-(alkylimino)bis[ethanol] described herein, including a combination described herein, e.g., with an acid and/or salt, may be formulated in compositions and consumer products (e.g., consumer products containing a composition described herein) that are used to counteract malodors in various air spaces and/or substrates.

### B. Malodor-Counteracting Compositions

The malodor-counteracting compounds described in Section I-A, including when in combination with an acid and/or salt as described herein, may be formulated in compositions that counteract malodor. Such compositions are generally referred to herein as malodor-counteracting compositions. It is contemplated that the malodor-counteracting compounds may be formulated into any composition that may be used to counteract malodor. For example, the compositions may include ingredients that are suitable for formulation into a consumer product, e.g., a consumer product as described in Section I-C. Thus, in some embodiments, the malodor-counteracting composition includes a malodor-counteracting compound described herein and ingredients suitable for formulation into consumer products.

As described supra, fragrance ingredients susceptible to hydrolysis, ethanolysis, solvolysis, retro-aldol elimination, dimerization, polymerization, and/or transesterification were found to be insensitive or have reduced sensitivity to degradation by the malodor-counteracting compounds described herein. Thus, in an aspect is provided a malodor-counteracting composition including a malodor-counteracting compound described herein and one or more fragrance ingredients susceptible to hydrolysis, ethanolysis, solvolysis, retro-aldol elimination, dimerization, polymerization, and/or transesterification. In some embodiments, the malodor-counteracting composition includes one or more fragrance ingredients susceptible to hydrolysis, ethanolysis, solvolysis, retro-aldol elimination, dimerization, polymerization, and/or transesterification, where the difference in degradation of the one or more fragrance ingredients in a condition including the malodor-counteracting compound and the same condition lacking the malodor-counteracting compound (control condition) is less than about 60%, 50%, 40%, 30%, 20%, 15%, 10%, 5%, 4%, 3%, 2%, or 1%. In some embodiments, the malodor-counteracting composition includes one or more fragrance ingredients susceptible to hydrolysis, ethanolysis, solvolysis, retro-aldol elimination, dimerization, polymerization, and/or transesterification, where the difference in degradation of the one or more fragrance ingredients in a condition including the malodor-counteracting compound and the same condition lacking the malodor-counteracting compound (control condition) is less than about 40%. In some embodiments, the malodor-counteracting composition includes one or more fragrance ingredients susceptible to hydrolysis, ethanolysis, solvolysis, retro-aldol elimination, dimerization, polymerization, and/or transesterification, where the difference in degradation of the one or more fragrance ingredients in a condition including the malodor-counteracting compound and the same condition lacking the malodor-counteracting compound (control condition) is less than about 20%. In some embodiments, the malodor-counteracting composition includes one or more fragrance ingredients susceptible to hydrolysis, ethanolysis, solvolysis, retro-aldol elimination, dimerization, polymerization, and/or transesterification, where the difference in degradation of the one or more fragrance ingredients in a condition including the malodor-counteracting compound and the same condition lacking the malodor-counteracting compound (control condition) is less than about 15%. In some embodiments, the malodor-counteracting composition includes one or more fragrance ingredients susceptible to hydrolysis, cthanolysis, solvolysis, retro-aldol elimination, dimerization, polymerization, and/or transesterification, where the difference in degradation of the one or more fragrance ingredients in a condition including the malodor-counteracting compound and the same condition lacking the malodor-counteracting compound (control condition) is less than about 10%. In some embodiments, the malodor-counteracting composition includes one or more fragrance ingredients susceptible to hydrolysis, ethanolysis, solvolysis, retro-aldol elimination, dimerization, polymerization, and/or transesterification, where the difference in degradation of the one or more fragrance ingredients in a condition including the malodor-counteracting compound and the same condition lacking the malodor-counteracting compound (control condition) is less than about 5%. In some embodiments, the condition is an accelerated aging condition as described herein. See, e.g., Section I-B-1 and Examples 6 and 7.

The malodor-counteracting compounds described herein may be formulated into a composition including ingredients with specific functions or activities. For example, a malodor-counteracting composition may further include malodor-counteracting ingredients, anti-microbial ingredients, ingredients with absorption or elimination properties, solvents, water absorbers, and/or stabilizers. Thus, in an aspect the malodor-counteracting composition includes a malodor-counteracting compound described herein and one or more ingredients with a specific function or activity.

In some embodiments, the malodor-counteracting compositions described herein include fragrance ingredients described herein and ingredients with a specific function or activity. It should be appreciated that individual ingredients described herein (e.g., fragrance ingredients, ingredients with a specific function or activity) or any combination of ingredients described herein (e.g., fragrance ingredients, ingredients with a specific function or activity) may be included in a malodor-counteracting composition.

### 1. Fragrance Ingredients

In some embodiments, the malodor-counteracting composition includes a 2,2′-(alkylimino)bis[ethanol] described herein and one or more fragrance ingredients. A fragrance ingredient may be alternatively referred to herein as a fragrance compound. In some embodiments, the fragrance ingredient is unstable at elevated temperature. In some embodiments, an elevated temperature is a temperature above room temperature. In some embodiments, the elevated temperature is a temperature between about room temperature and about 50° C. In some embodiments, the elevated temperature is a temperature between about room temperature and about 30° C. In some embodiments, the fragrance ingredient is unstable during prolonged storage. In some embodiments, prolonged storage is storage for a duration of or greater than 1 week. In some embodiments, prolonged storage is for a duration of or greater than 1 month. In some embodiments, prolonged storage is for a duration of or greater than 2 months.

In some embodiments, the fragrance ingredient is susceptible to hydrolysis, cthanolysis, solvolysis, retro-aldol elimination, dimerization, polymerization, and/or transesterification. In some cases, the susceptibility increases at elevated temperature or during prolonged storage, as described herein.

In some embodiments, the fragrance ingredient susceptible to hydrolysis, ethanolysis, solvolysis, retro-aldol elimination, dimerization, polymerization, and/or transesterification degrades by less than about 60%, 50%, 40%, 30%, 20%, 15%, 10%, 5%, 4%, 3%, 2%, or 1% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades by less than about 50% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades by less than about 40% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades by less than about 30% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades by less than about 20% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades by less than about 10% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades by less than about 5% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades by less than about 4% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades by less than about 3% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades by less than about 2% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades by less than about 1% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades in a range of about 0% to 50% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades in a range of about 0% to 40% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades in a range of about 0% to 30% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades in a range of about 0% to 20% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, cthanolysis, solvolysis, retro-aldol elimination, dimerization, polymerization, and/or transesterification degrades in a range of about 0% to 10% in the presence of a 2,2′-(alkylimino)bis[ethanol] described herein compared to the degradation of the fragrance ingredient in a control condition lacking the 2,2′-(alkylimino)bis[ethanol]. In some embodiments, the fragrance ingredient susceptible to hydrolysis, ethanolysis, solvolysis, retro-aldol elimination, dimerization, polymerization, and/or transesterification degrades in a range of about 0% to 5% in the presence of a 2,2′-(alkylimino)bis[ethanol] described herein compared to the degradation of the fragrance ingredient in a control condition lacking the 2,2′-(alkylimino)bis[ethanol]. It should be appreciated that the control condition is identical in all aspects to the condition that includes the 2,2′-(alkylimino)bis[ethanol], the only difference between the conditions is the presence or absence (control) of the 2,2′-(alkylimino)bis[ethanol]. In this way, the difference in degradation between the two conditions may be compared to determine the percentage of degradation of the fragrance ingredient in the presence of 2,2′-(alkylimino)bis[ethanol]. By way of example, the degradation of the fragrance ingredient in the control condition may serve as a baseline for the fragrance ingredient degradation, and the degradation observed in the condition including 2,2′-(alkylimino)bis[ethanol] may be quantified, e.g., as a percent, relative to the baseline. In some embodiments, the condition is an accelerated aging condition. Any accelerated aging condition may be used to assess the amount of degradation of the fragrance ingredient. In some embodiments, the accelerated aging condition includes 4 weeks of storage at 40° C. In some embodiments, the accelerated aging condition includes 4 weeks of storage at 40° C. in an ambient environment. For example, an ambient environment does not exclude air from the samples. In some embodiments, the degradation is determined using gas chromatography-mass spectrometry (GC-MS). For example, see, Examples 6 and 7 below. In some embodiments, the degradation is determined by a sensory panelist. In some embodiments, the degradation is determined by a change in smell. In some embodiments, amount of degradation in the condition is determined by a change in color. The degradation may be quantified to allow for further analyses, such as, a comparison of degradation across conditions. In some embodiments, the quantified change in smell, color, and/or other sensory characteristic is less than 40%, 30%, 20%, 15%, 10%, 5%, 4%, 3%, 2%, or 1%. In some embodiments, the quantified change in smell, color, and/or other sensory characteristic is less than 20%. In some embodiments, the quantified change in smell, color, and/or other sensory characteristic is less than 15%. In some embodiments, the quantified change in smell, color, and/or other sensory characteristic is less than 10%. In some embodiments, the quantified change in smell, color, and/or other sensory characteristic is less than 5%.

In some embodiments, the fragrance ingredient is an aldehyde, an aldehyde precursor, an ester, an ester precursor, a lactone, or a lactone precursor. As described above, a precursor as used herein refers to a compound that when formulated into an accord, full fragrance, or consumer product will convert to an aldehyde, an ester, or a lactone as a result of a chemical reaction. In some embodiments, the aldehyde, the aldehyde precursor, the ester, the ester precursor, the lactone, and/or the lactone precursor is susceptible to hydrolysis, ethanolysis, solvolysis, retro-aldol climination, dimerization, polymerization, and/or transesterification.

In some embodiments, the fragrance ingredient is an aldehyde. In some embodiments, the aldehyde has the formula:

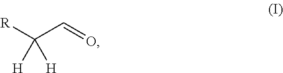

where: R is a C₁₋₁₀ alkyl, C₁₋₁₀ alkenyl, or C₁₋₁₀ alkynyl. In some embodiments, R is a C₁₋₁₀ alkyl. In some embodiments, R is a C₁₋₁₀ alkenyl. In some embodiments, R is a C₁₋₁₀ alkynyl. In some embodiments, the R group is substituted or unsubstituted. In some embodiments, the R group is substituted by one or more of a hydroxyl, an ester, an ether, a cyclopentyl, a cyclohexyl, a cyclopentadienyl, a benzyl, or a furanyl group. In some embodiments, the R group is substituted by a hydroxyl group. In some embodiments, the R group is substituted by an ester group. In some embodiments, the R group is substituted by an ether group. In some embodiments, the R group is substituted by a cyclopentyl group. In some embodiments, the R group is substituted by a cyclohexyl group. In some embodiments, the R group is substituted by a cyclopentadienyl group. In some embodiments, the R group is substituted by a benzyl group. In some embodiments, the R group is substituted by a furanyl group. In some embodiments, the aldehyde is octanal, nonanal, decanal, 10-undecenal, or dodecanal. In some embodiments, the aldehyde is octanal. In some embodiments, the aldehyde is nonanal. In some embodiments, the aldehyde is decanal. In some embodiments, the aldehyde is 10-undecenal. In some embodiments, the aldehyde is dodecanal. In some embodiments, the aldehyde degrades by less than about 60%, 50%, 40%, 30%, 20%, 15%, 10%, 5%, 4%, 3%, 2%, or 1% in the presence of a 2,2′-(alkylimino)bis[ethanol] described herein compared to the degradation of the aldehyde in a control condition lacking the 2,2′-(alkylimino)bis[ethanol]. In some embodiments, the aldehyde degrades by less than about 50% in the presence of a 2,2′-(alkylimino)bis[ethanol] described herein compared to the degradation of the aldehyde in a control condition lacking the 2,2′-(alkylimino)bis[ethanol]. In some embodiments, the aldehyde degrades by less than about 40% in the presence of a 2,2′-(alkylimino)bis[ethanol] described herein compared to the degradation of the aldehyde in a control condition lacking the 2,2′-(alkylimino)bis[ethanol]. In some embodiments, the aldehyde degrades by less than about 20% in the presence of a 2,2′-(alkylimino)bis[ethanol] described herein compared to the degradation of the aldehyde in a control condition lacking the 2,2′-(alkylimino)bis[ethanol]. In some embodiments, the aldehyde degrades by less than about 10% in the presence of a 2,2′-(alkylimino)bis[ethanol] described herein compared to the degradation of the aldehyde in a control condition lacking the 2,2′-(alkylimino)bis[ethanol]. In some embodiments, the aldehyde degrades by less than about 5% in the presence of a 2,2′-(alkylimino)bis[ethanol] described herein compared to the degradation of the aldehyde in a control condition lacking the 2,2′-(alkylimino)bis[ethanol].

In some embodiments, the fragrance ingredient is an ester. In some embodiments, the ester is derived from cis-3-hexenyl alcohol or a derivative thereof, benzyl alcohol or a derivative thereof, allyl alcohol or a derivative thereof, or benzoic acid or a derivative thereof. In some embodiments, the ester is derived from: p-methylbenzyl alcohol, cuminyl alcohol, cinnamyl alcohol, prenyl alcohol, geraniol/nerol, farnesol, or salicylic acid. In some embodiments, the ester is benzyl acetate, cis-3-hexenyl acetate, geranyl acetate, hexyl salicylate, cinnamyl acetate, (4-(prop-1-en-2-yl)cyclohex-1-en-1-yl)methyl acetate (dihydrocuminyl acetate), allyl 2-(cyclohexyloxy)acetate (Cyclogalbanate), 3-methylbut-2-en-1-yl acetate (prenyl acetate), anisyl acetate, benzyl butyrate, benzyl cinnamate, benzyl propionate, benzyl salicylate, 4-isopropylbenzyl acetate (cuminyl acetate), para-methylbenzyl acetate, amyl salicylate, cis-3-hexenyl salicylate, ethyl salicylate, methyl salicylate, methyl 2-(3-oxo-2-pentylcyclopentyl)acetate (methyl dihydrojasmonate), allyl caproate, allyl caprylate, farnesyl acetate, geranyl acetate, geranyl propionate, neryl acetate, benzyl benzoate, benzyl iso butyrate, or methyl 4-methoxybenzoate (methyl anisate). In some embodiments, the ester is benzyl acetate. In some embodiments, the ester is cis-3-hexenyl acetate. In some embodiments, the ester is geranyl acetate. In some embodiments, the ester is hexyl salicylate. In some embodiments, the ester is cinnamyl acetate. In some embodiments, the ester is (4-(prop-1-en-2-yl)cyclohex-1-en-1-yl)methyl acetate (dihydrocuminyl acetate). In some embodiments, the ester is allyl 2-(cyclohexyloxy)acetate (Cyclogalbanate). In some embodiments, the ester is 3-methylbut-2-en-1-yl acetate (prenyl acetate). In some embodiments, the ester is anisyl acetate. In some embodiments, the ester is benzyl butyrate. In some embodiments, the ester is benzyl cinnamate. In some embodiments, the ester is benzyl propionate. In some embodiments, the ester is benzyl salicylate. In some embodiments, the ester is 4-isopropylbenzyl acetate (cuminyl acetate). In some embodiments, the ester is para-methylbenzyl acetate. In some embodiments, the ester is amyl salicylate. In some embodiments, the ester is cis-3-hexenyl salicylate. In some embodiments, the ester is ethyl salicylate. In some embodiments, the ester is methyl salicylate. In some embodiments, the ester is methyl 2-(3-oxo-2-pentylcyclopentyl)acetate (methyl dihydrojasmonate). In some embodiments, the ester is allyl caproate. In some embodiments, the ester is allyl caprylate. In some embodiments, the ester is farnesyl acetate. In some embodiments, the ester is geranyl acetate. In some embodiments, the ester is geranyl propionate. In some embodiments, the ester is neryl acetate. In some embodiments, the ester is benzyl benzoate. In some embodiments, the ester is benzyl iso butyrate. In some embodiments, the ester is methyl 4-methoxybenzoate (methyl anisate). In some embodiments, the ester degrades by less than about 60%, 50%, 40%, 30%, 20%, 15%, 10%, 5%, 4%, 3%, 2%, or 1% in the presence of a 2,2′-(alkylimino)bis[ethanol] described herein compared to the degradation of the ester in a control condition lacking the 2,2′-(alkylimino)bis[ethanol]. In some embodiments, the ester degrades by less than about 50% in the presence of a 2,2′-(alkylimino)bis[ethanol] described herein compared to the degradation of the ester in a control condition lacking the 2,2′-(alkylimino)bis[ethanol]. In some embodiments, the ester degrades by less than about 40% in the presence of a 2,2′-(alkylimino)bis[ethanol] described herein compared to the degradation of the ester in a control condition lacking the 2,2′-(alkylimino)bis[ethanol]. In some embodiments, the ester degrades by less than about 20% in the presence of a 2,2′-(alkylimino)bis[ethanol] described herein compared to the degradation of the ester in a control condition lacking the 2,2′-(alkylimino)bis[ethanol]. In some embodiments, the ester degrades by less than about 10% in the presence of a 2,2′-(alkylimino)bis[ethanol] described herein compared to the degradation of the ester in a control condition lacking the 2,2′-(alkylimino)bis[ethanol]. In some embodiments, the ester degrades by less than about 5% in the presence of a 2,2′-(alkylimino)bis[ethanol] described herein compared to the degradation of the ester in a control condition lacking the 2,2′-(alkylimino)bis[ethanol].

In some embodiments, the fragrance ingredient is a lactone. In some embodiments, the lactone is a macrocycle including at least 15 atoms in a ring and a substructure having the formula:

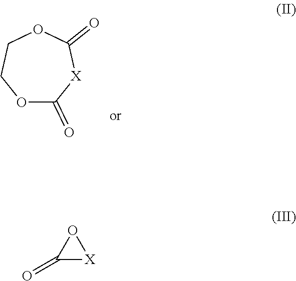

wherein: X is a saturated or unsaturated alkyl chain including 0-1methyl groups. In some embodiments, X is saturated. In some embodiments, X is an unsaturated alkyl chain including 0-1 methyl groups. In some embodiments, the lactone is ethylene brassylate. In some embodiments, the lactone is (E)-oxacycloheptadec-10-en-2-one (ambrettolide). In some embodiments, the lactone degrades by less than about 60%, 50%, 40%, 30%, 20%, 15%, 10%, 5%, 4%, 3%, 2%, or 1% in the presence of a 2,2′-(alkylimino)bis[ethanol] described herein compared to the degradation of the lactone in a control condition lacking the 2,2′-(alkylimino)bis[ethanol]. In some embodiments, the lactone degrades by less than about 50% in the presence of a 2,2′-(alkylimino)bis[ethanol] described herein compared to the degradation of the lactone in a control condition lacking the 2,2′-(alkylimino)bis[ethanol]. In some embodiments, the lactone degrades by less than about 40% in the presence of a 2,2′-(alkylimino)bis[ethanol] described herein compared to the degradation of the lactone in a control condition lacking the 2,2′-(alkylimino)bis[ethanol]. In some embodiments, the lactone degrades by less than about 20% in the presence of a 2,2′-(alkylimino)bis[ethanol] described herein compared to the degradation of the lactone in a control condition lacking the 2,2′-(alkylimino)bis[ethanol]. In some embodiments, the lactone degrades by less than about 10% in the presence of a 2,2′-(alkylimino)bis[ethanol] described herein compared to the degradation of the lactone in a control condition lacking the 2,2′-(alkylimino)bis[ethanol]. In some embodiments, the lactone degrades by less than about 5% in the presence of a 2,2′-(alkylimino)bis[ethanol] described herein compared to the degradation of the lactone in a control condition lacking the 2,2′-(alkylimino)bis[ethanol].

In some embodiments, the fragrance ingredient is a phenethyl alcohol, tetrahydrolinalool, linalool, 3-phenylpropanal, cinnamaldehyde, decanal, ethyl linalool, dihydromyrcenol, 2-methyldecanal, (3E)-4-Methyl-3-decen-5-one, or 4-Methyl-3-decen-5-ol (undecavertol). In some embodiments, the fragrance ingredient is a phenethyl alcohol. In some embodiments, the fragrance ingredient is tetrahydrolinalool. In some embodiments, the fragrance ingredient is linalool. In some embodiments, the fragrance ingredient is 3-phenylpropanal, cinnamaldehyde. In some embodiments, the fragrance ingredient is decanal. In some embodiments, the fragrance ingredient is ethyl linalool. In some embodiments, the fragrance ingredient is dihydromyrcenol. In some embodiments, the fragrance ingredient is 2-methyldecanal. In some embodiments, the fragrance ingredient is (3E)-4-Methyl-3-decen-5-one. In some embodiments, the fragrance ingredient is 4-Methyl-3-decen-5-ol (undecavertol).

In some cases, the malodor-counteracting composition may include 1, 2, 3, 4, 5, 10, 20, 30, 40, 50 100, or more fragrance ingredients. The ratio of a malodor-counteracting compound to fragrance ingredient may be expressed as the ratio of the malodor-counteracting compound to a single ingredient, regardless of whether there is only one or more fragrance ingredients in the composition. For example, in some embodiments, the ratio of malodor-counteracting compound to a single fragrance ingredient is at least 1:0.5. In some embodiments, the ratio of malodor-counteracting compound to a single fragrance ingredient is at least 1:1. In some embodiments, the ratio of malodor-counteracting compound to a single fragrance ingredient is in a range of about 1:0.5 to 1000:1. In some embodiments, the ratio of malodor-counteracting compound to a single fragrance ingredient is 100:1, 500:1, or 1000:1. The ratio of the malodor-counteracting compound may also be expressed as the ratio of the malodor-counteracting compound to all fragrance ingredients present in the malodor-counteracting composition. For example, in some embodiments, the ratio of malodor-counteracting compound to all fragrance ingredients is in a range of about 1:50 to 10:1. In some embodiments, the ratio of malodor-counteracting compound to all fragrance ingredients is in a range of about 1:20 to 10:1. In some embodiments, the ratio of malodor-counteracting compound to all fragrance ingredients is in a range of about 1:10 to 10:1. In some embodiments, the ratio of malodor-counteracting compound to all fragrance ingredients is in a range of about 1:5 to 10:1. In some embodiments, the ratio of malodor-counteracting compound to all fragrance ingredients is 1:50, 1:20, 1:10, 1:1, 5:1, or 10:1. In some embodiments, the ratio of malodor-counteracting compound to all fragrance ingredients is 1:10, 1:5, 1:2, 2:1, 5:1, or 10:1. In some embodiments, the ratio is a weight ratio.

In some embodiments, the fragrance ingredient is included in a natural oil. In some embodiments, the fragrance ingredient is included in an accord. In some embodiments, the fragrance ingredient is included in a full fragrance. In some embodiments, the fragrance ingredient, optionally included in a natural oil, accord, or a full fragrance, is contained in a capsule. Thus, in some embodiments, the fragrance ingredient is encapsulated.

Capsules, e.g., microcapsules, may be used to deliver, apply, or release a fragrance ingredient to a target area in a time-delayed or controlled manner. In some embodiments, the capsules are sustainable capsules. In some embodiments, the capsules are microcapsules. In some embodiments, the capsule includes a polymer. For example, the capsule wall is formed by a polymer. In some embodiments, the polymer is polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde) and a combination thereof. Non-limiting examples of capsules, methods of making capsules, and methods of encapsulating ingredients (e.g., fragrance ingredients) are described in published applications WO2020/131890, WO2020/131866, WO2018053356, US2019/0076811, US2022/0226208, WO2018/006089, WO2019/227019, WO2020/131956, WO2020/131875, WO2020/131879, WO2015/070228, and WO2017/192648, which are incorporated herein by reference in their entirety. In some embodiments, the capsule further includes a 2,2′-(alkylimino)bis[ethanol] described herein. Thus, in some cases, the 2,2′-(alkylimino)bis[ethanol] described herein and the fragrance ingredient, optionally included in a natural oil, accord, or a full fragrance, are contained in the same capsule. In some embodiments, the 2,2′-(alkylimino)bis[ethanol] described herein and the fragrance ingredient, optionally included in a natural oil, accord, or a full fragrance, are contained in separate capsules. In some embodiments, the separate capsules include the same polymer or are identical in formulation. In some embodiments, the separate capsules include different polymers or are different in formulation.

In some embodiments, the malodor-counteracting composition includes one or more fragrance ingredients described herein. In some embodiments, the malodor-counteracting composition includes one or more fragrance ingredients described herein in an encapsulated form. In some embodiments, the malodor-counteracting compound included in the malodor-counteracting composition is 2,2′-(octylimino) bis [ethanol].

### 2. Functional and Active Ingredients

In some embodiments, the malodor-counteracting composition includes a 2,2′-(alkylimino)bis[ethanol] described herein and an ingredient having a function or activity. In some embodiments, the functional or active ingredient is one or more of a solvent, an antibacterial active, a water absorber, an absorption or elimination active, a stabilizer, an olfactory receptor blocker, or a volatile organic chemical affecting fragrance perception. In some embodiments, the functional or active ingredient is one or more of a solvent, an antibacterial active, a water absorber, an absorption or elimination active, a stabilizer, or an olfactory receptor blocker.

In some embodiments, the malodor-counteracting composition includes a 2,2′-(alkylimino)bis[ethanol] described herein and a solvent. In some embodiment, the solvent is suitable for use with fragrance ingredients. Non-limiting examples of solvents include isopropyl myristate (IPM), dipropylene glycol (DPG), triethyl citrate, triacetin, IPP, IPL, hercolyn, dowanol, neobec, isopar, propylene glycol, and benz benzoate. In some embodiments, the solvent is IPM, DPG, tricthyl citrate, or a combination thereof. In some embodiments, the solvent is isopropyl myristate (IPM). In some embodiments, the solvent is dipropylene glycol (DPG). In some embodiments, the solvent is triethyl citrate.

In some embodiments, the malodor-counteracting composition includes a 2,2′-(alkylimino)bis[ethanol] as describe herein and a deodorizing agent. In some embodiments, the deodorizing agent is a salt. Any type of salt known to deodorize is contemplated for use herein. In some embodiments, the salt is a zinc salt. In some embodiments, the salt is zinc phenolsulfonate. In some embodiments, the salt is zinc ricinoleate. In some embodiments, the salt is zinc neodecanoate. In some embodiments, the salt is zinc stearate. In some embodiments, the salt is zinc bound to a polymer. In some embodiments, the polymer is a polyitaconate or a functionalized silicone. In some embodiments, the salt is not a zinc salt.

In some embodiments, the malodor-counteracting composition includes a 2,2′-(alkylimino)bis[ethanol] described herein and an ingredient for eliminating reactive malodor molecules. For example, the ingredient may form a covalent bond with the malodor molecule to change the negative sensory properties of the malodor molecule. In some embodiments, the malodor-counteracting composition includes a 2,2′-(alkylimino)bis[ethanol] described herein and an unsaturated carbonyl compound (e.g., alpha,beta-unsaturated esters or ketones), an esters of a phenolic compound, a rings containing oxygen (e.g., epoxides, cyclic carbonate) or a combination thereof.

In some embodiments, the malodor-counteracting composition includes a 2,2′-(alkylimino)bis[ethanol] described herein and an antibacterial active. Non-limiting examples of antibacterial actives include antibacterial alcohols, bactericidal acids, enzymes, botanical extracts, diols and polyols, quaternary ammonium compounds, peptides, silver metals and silver salts, formaldehyde releasing compounds and halogenated compounds.

In some embodiments, the malodor-counteracting composition includes a 2,2′-(alkylimino)bis[ethanol] described herein and an absorption or elimination active. Non-limiting examples of absorption and elimination actives include activated carbons cyclodextrins, diatomaceous earth, metal oxides, polymeric amines, and organic or inorganic active oxygen.

In some embodiments, the malodor-counteracting composition includes a 2,2′-(alkylimino)bis[ethanol] described herein and a water absorber. Non-limiting examples of water absorbers include clay, aluminum salt, magnesium oxide, talc, polyacrylate, cellulose, and magnesium sulfate.

In some embodiments, the malodor-counteracting composition includes a 2,2′-(alkylimino)bis[ethanol] described herein and an olfactory receptor blocker. Non-limiting examples of olfactory receptor blockers include thiols, sulfides, indole, and carboxylic acids.

In some embodiments, the malodor-counteracting composition includes a 2,2′-(alkylimino)bis[ethanol] described herein and a stabilizer. In some embodiments, the stabilizer preserves the malodor-counteracting composition. In some embodiments, the stabilizer is a fragrance stabilizer. For example, fragrance stabilizers may be included when the composition further includes fragrance ingredients. Non-limiting examples of stabilizers, including fragrance stabilizers, include UV filters, antioxidants, and chelating agents.

In some embodiments, malodor-counteracting composition includes a 2,2′-(alkylimino)bis[ethanol] described herein and volatile organic chemicals affecting fragrance perception. Non-limiting examples of volatile organic chemicals affecting fragrance perception may be found in published international application WO2017/046055, which is incorporated herein by reference in its entirety.

Any combination of functional or active ingredient is contemplated as useful for the malodor-counteracting composition. It should be appreciated that a fragrance ingredient, for example as described in Section I-B-1, may also be included in malodor-counteracting compositions including functional and/or active ingredients.

### 3. Other Materials

Other materials can also be used in conjunction with the malodor-counteracting compositions to encapsulate and/or deliver the compositions. Some well-known materials are, for example, but not limited to, polymers, oligomers, other non-polymers such as surfactants, emulsifiers, lipids including fats, waxes and phospholipids, organic oils, mineral oils, petrolatum, natural oils, perfume fixatives, fibers, starches, sugars and solid surface materials such as zeolite and silica. Some preferred polymers include polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde), or a combination thereof.

### 4. Concentrations of Malodor-Counteracting Compounds

The malodor-counteracting compounds described herein are contemplated as a being present in compositions described herein in amounts (e.g., concentrations) that are effective in counteracting malodor. An effective amount is understood to mean the amount of a malodor-counteracting compound described herein that is organoleptically effective to abate a given malodor. In some embodiments, the malodor-counteracting compositions may be added to consumer products. Thus, in some embodiments, the effective amount of the malodor-counteracting compound is present at a level that can be formulated into a consumer product to reach an effective concentration. For example, the concentration of the malodor-counteracting compound in the malodor-counteracting composition may be higher than the effective amount useful in the consumer product.

In some embodiments, the malodor-counteracting compound in the malodor-counteracting composition is present in an amount of at least or about 0.005, 0.01, 0.1, 0.5, 1, 2, 3, 4, 5, 10, 20, 30, 40, 50, 60, 70, 80, 90, or 100% wt. In some embodiments, the malodor-counteracting compound in the malodor-counteracting composition is present in an amount of at least or about 0.005, 0.01, 0.1, 0.5, 1, 2, 3, 4, 5, 5, 6, 7, 9, or 10% wt. In some embodiments, the malodor-counteracting compound in the malodor-counteracting composition is present in an amount of at least or about 0.005, 0.01, 0.1, 0.5, 1, 2, 3, 4, or 5% wt. In some embodiments, the malodor-counteracting compound in the malodor-counteracting composition is present in an amount ranging from about 0.005% wt to about 10% wt. In some embodiments, the malodor-counteracting compound in the malodor-counteracting composition is present in an amount ranging from about 0.01% wt to about 9% wt. In some embodiments, the malodor-counteracting compound in the malodor-counteracting composition is present in an amount ranging from about 0.01% wt to about 8% wt. In some embodiments, the malodor-counteracting compound in the malodor-counteracting composition is present in an amount ranging from about 0.01% wt to about 7% wt. In some embodiments, the malodor-counteracting compound in the malodor-counteracting composition is present in an amount ranging from about 0.01% wt to about 6% wt. In some embodiments, the malodor-counteracting compound in the malodor-counteracting composition is present in an amount ranging from about 0.01% wt to about 5% wt. In some embodiments, the malodor-counteracting compound in the malodor-counteracting composition is present in an amount ranging from about 0.01% wt to about 4% wt. In some embodiments, the malodor-counteracting compound in the malodor-counteracting composition is present in an amount ranging from about 0.01% wt to about 3% wt. In some embodiments, the malodor-counteracting compound in the malodor-counteracting composition is present in an amount ranging from about 0.01% wt to about 2% wt. In some embodiments, the malodor-counteracting compound in the malodor-counteracting composition is present in an amount ranging from about 0.01% wt to about 1% wt. In some embodiments, the malodor-counteracting compound in the malodor-counteracting composition is present in an amount ranging from about 0.01% wt to about 0.5% wt.

In some embodiments, the malodor-counteracting compound in the malodor-counteracting composition is present in an amount of at least or about 60, 70, 80, 90, or 100% wt. In some embodiments, the malodor-counteracting compound in the malodor-counteracting composition is present in an amount of at least or about 70, 80, 90, or 100% wt. In some embodiments, the malodor-counteracting compound in the malodor-counteracting composition is present in an amount ranging from about 70% wt to about 100% wt. In some embodiments, the malodor-counteracting compound in the malodor-counteracting composition is present in an amount ranging from about 75% wt to about 100% wt. In some embodiments, the malodor-counteracting compound in the malodor-counteracting composition is present in an amount ranging from about 80% wt to about 100% wt. In some embodiments, the malodor-counteracting compound in the malodor-counteracting composition is present in an amount ranging from about 85% wt to about 100% wt. In some embodiments, the malodor-counteracting compound in the malodor-counteracting composition is present in an amount ranging from about 90% wt to about 100% wt. In some embodiments, the malodor-counteracting compound in the malodor-counteracting composition is present in an amount ranging from about 95% wt to about 100% wt. In some embodiments, the malodor-counteracting compound is 95, 96, 97, 98, 99, or 100% wt of the malodor-counteracting composition. In some embodiments, the malodor-counteracting compound is 100% wt of the malodor-counteracting composition.

### C. Consumer Products

The malodor-counteracting compounds and malodor-counteracting compositions may be included in or formulated for use in consumer products. These consumer products may be referred to alternatively as functional products.

Non-limiting examples of the consumer products provided herein include, for example, a conventional room freshener (or deodorant) composition such as room freshener sprays, an aerosol or other spray, fragrance diffusers, a wick or other liquid system, or a solid, for instance candles or a wax base as in pomanders and plastics, powders as in sachets or dry sprays or gels, as in solid gel sticks, clothes deodorants as applied by washing machine applications such as in detergents, powders, liquids, whiteners or fabric softeners, fabric refreshers, linen sprays, closet blocks, closet aerosol sprays, or clothes storage areas or in dry cleaning to overcome residual solvent notes on clothes, bathroom accessories such as paper towels, bathroom tissues, sanitary napkins, towellets, disposable wash cloths, disposable diapers, and diaper pail deodorants, cleansers such as disinfectants and toilet bowl cleaners, cosmetic products such as antiperspirant and deodorants, general body deodorants in the form of powders, aerosols, liquids or solid, feminine care products such as tampons and feminine napkins, baby care products such as diapers, bibs and wipes, or hair care and treatment products such as hair sprays, conditioners, shampoos, rinses, hair colors and dyes, permanent waves, depilatories, hair straighteners, hair groom applications such as pomade, creams and lotions, medicated hair care products containing such ingredients as selenium sulphide, coal tar or salicylates, or shampoos, or foot care products such as foot powders, liquids or colognes, after shaves and body lotions, or soaps and synthetic detergents such as bars, liquids, foams or powders, odor control such as during manufacturing processes, such as in the textile finishing industry and the printing industry (inks and paper), effluent control such as in processes involved in pulping, stock yard and meat processings, sewage treatment, garbage bags, or garbage disposal, or in product odor control as in textile finished goods, rubber finished goods or car fresheners, agricultural and pet care products such as dog and hen house effluents and domestic animal and pet care products such as deodorants, shampoo or cleaning agents, or animal litter material and in large scale closed air systems such as auditoria, and subways and transport systems.

Thus, it should be appreciated that the malodor-counteracting compound or malodor-counteracting composition may be present together with a carrier by means of which or from which the malodor-counteracting compound can be introduced into an air space wherein the malodor is present, or a substrate on which the malodor has deposited. For example, the carrier can be an aerosol propellant such as a chlorofluoro-methane, or a solid such as a wax, plastics material, rubber, inert powder or gel. Aerosol propellants may be hydrocarbon or halogenated hydrocarbon gas, such as fluorinated hydrocarbons such as 1,1-difluoroethane and/or 1-trifluoro-2-fluorocthane. In some embodiments, the propellant includes liquefied hydrocarbon gases, and C3 to C5 hydrocarbons, including propane, isopropane, butane, isobutane, pentane and isopentane and mixtures of two or more thereof. In some embodiments, the propellants are isobutane, isobutane/isopropane, isobutane/propane and mixtures of isopropane, isobutane and butane. In a wick-type air freshener, the carrier is a substantially odorless liquid of low volatility. In some embodiments, the consumer product or malodor-counteracting composition contains a surface active agent or a disinfectant, while in others, the malodor counteractant is present on a fibrous substrate. In some embodiments, the consumer product includes a fragrance component which imparts a fragrance. Many types of fragrances can be employed in the present invention, the only limitation being the compatibility with the other components being employed. Suitable fragrances include but are not limited to fruits such as almond, apple, cherry, grape, pear, pineapple, orange, strawberry, raspberry; musk, flower scents such as lavender-like, rose-like, iris-like, carnation-like. Other pleasant scents include herbal and woodland scents derived from pine, spruce and other forest smells. Fragrances may also be derived from various oils, such as essential oils, or from plant materials such as peppermint, spearmint and the like. A list of suitable fragrances is provided in U.S. Pat. No. 4,534,891, the contents of which are incorporated by reference as if set forth in its entirety. Fragrances contemplated for use herein are also described in U.S. Pat. Nos. 5,683,979, 6,379,658, 6,432,891, the contents of which are incorporated by reference as if set forth in its entirety. Another source of suitable fragrances is found in Perfumes, Cosmetics and Soaps, Second Edition, edited by W. A. Poucher, 1959. Among the fragrances provided in this treatise are acacia, cassie, chypre, cyclamen, fern, gardenia, hawthorn, heliotrope, honeysuckle, hyacinth, jasmine, lilac, lily, magnolia, mimosa, narcissus, freshly-cut hay, orange blossom, orchid, reseda, sweet pea, trefle, tuberose, vanilla, violet, wallflower, and the like. In some embodiments, a fragrance ingredient as described herein (see, Section I-B-1) is included in the consumer product.

### 1. Malodor Effective Amounts

The malodor-counteracting compound described herein is contemplated as being present in consumer products described herein in amounts (e.g., concentrations) that are effective in counteracting malodor. As described above, an effective amount is understood to mean the amount of a malodor-counteracting compound described herein that is organoleptically effective to abate a given malodor. The malodor to be counteracted may be present in air space or on a substrate. It should be appreciated that the exact amount of malodor-counteracting compound needed to be an effective amount may vary depending upon the type of the type of malodor, the consumer product, and the level of malodor counteractancy desired. In general, the amount of malodor-counteracting compound present is the ordinary dosage required to obtain the desired result.

In some embodiments, the malodor-counteracting compound is present in a consumer product at a concentration of at least about 0.005% wt of the consumer product. In some embodiments, the malodor-counteracting compound is present in the consumer product at a concentration in a range of about 0.005% wt to about 10% wt of the consumer product. In some embodiments, the malodor-counteracting compound is present in the consumer product at a concentration in a range of about 0.005% wt to about 5% wt of the consumer product. In some embodiments, the malodor-counteracting compound is present in the consumer product at a concentration in a range of about 0.005% wt to about 4% wt of the consumer product. In some embodiments, the malodor-counteracting compound is present in the consumer product at a concentration in a range of about 0.005% wt to about 3% wt of the consumer product. In some embodiments, the malodor-counteracting compound is present in the consumer product at a concentration in a range of about 0.005% wt to about 2% wt of the consumer product. In some embodiments, the malodor-counteracting compound is present in the consumer product at a concentration in a range of about 0.005% wt to about 1% wt of the consumer product. In some embodiments, the malodor-counteracting compound is present in the consumer product at a concentration in a range of about 0.01% wt to about 10% wt of the consumer product. In some embodiments, the malodor-counteracting compound is present in the consumer product at a concentration in a range of about 0.1% wt to about 10% wt of the consumer product. In some embodiments, the malodor-counteracting compound is present in the consumer product at a concentration in a range of about 0.5% wt to about 10% wt of the consumer product. In some embodiments, the malodor-counteracting compound is present in the consumer product at a concentration in a range of about 1% wt to about 10% wt of the consumer product. In some embodiments, the malodor-counteracting compound is present in the consumer product at a concentration in a range of about 2% wt to about 10% wt of the consumer product. In some embodiments, the malodor-counteracting compound is present in the consumer product at a concentration in a range of about 3% wt to about 10% wt of the consumer product. In some embodiments, the malodor-counteracting compound is present in the consumer product at a concentration in a range of about 4% wt to about 10% wt of the consumer product. In some embodiments, the malodor-counteracting compound is present in the consumer product at a concentration in a range of about 5% wt to about 10% wt of the consumer product. In some embodiments, the malodor-counteracting compound is present in the consumer product at a concentration in a range of about 0.5% wt to about 5% wt of the consumer product. In some embodiments, the malodor-counteracting compound is present in the consumer product at a concentration in a range of about 1% wt to about 5% wt of the consumer product. In some embodiments, the malodor-counteracting compound is present in the consumer product at a concentration in a range of about 0.5% wt to about 2% wt of the consumer product.

In some embodiments, for example when used in consumer products such as an underarm deodorant or antiperspirant, the malodor-counteracting compound described herein may be present in an amount ranging from about 0.1% wt to about 5% wt. In some embodiments, for example when used in consumer products such as an underarm deodorant or antiperspirant, the malodor-counteracting compound described herein may be present in an amount ranging from about 0.5% wt to about 2% wt.

In some embodiments, for example when used in consumer products such as an eye product, baby cream, face cream, a body lotion, a hair styling spray, a scent strip, a facial make-up, a hand cream, a wipe or tissue, a powder or talc, or a hair dye, the malodor-counteracting compound described herein may be present in an amount ranging from about 0.1% wt to about 2% wt. In some embodiments, for example when used in consumer products such as an eye product, baby cream, face cream, a body lotion, a hair styling spray, a scent strip, a facial make-up, a hand cream, a wipe or tissue, a powder or talc, or a hair dye, the malodor-counteracting compound described herein may be present in an amount ranging from about 0.1% wt to about 1% wt. In some embodiments, for example when used in consumer products such as an eye product, baby cream, face cream, a body lotion, a hair styling spray, a scent strip, a facial make-up, a hand cream, a wipe or tissue, a powder or talc, or a hair dye, the malodor-counteracting compound described herein may be present in an amount ranging from about 0.1% wt to about 0.5% wt. In some embodiments, for example when used in consumer products such as an eye product, baby cream, face cream, a body lotion, a hair styling spray, a scent strip, a facial make-up, a hand cream, a wipe or tissue, a powder or talc, or a hair dye, the malodor-counteracting compound described herein may be present in an amount of about 0.1% wt.

In some embodiments, for example when used in consumer products such as a personal wash (e.g., soap, shower gel) a shampoo, a shaving product, or an aerosol air care, the malodor-counteracting compound described herein may be present in an amount ranging from about 0.01% wt to about 5% wt. In some embodiments, for example when used in consumer products such as a personal wash (e.g., soap, shower gel) a shampoo, a shaving product, or an aerosol air care, the malodor-counteracting compound described herein may be present in an amount ranging from about 0.5% wt to about 5% wt. In some embodiments, for example when used in consumer products such as a personal wash (e.g., soap, shower gel) a shampoo, a shaving product, or an aerosol air care, the malodor-counteracting compound described herein may be present in an amount of about 1% wt.

In some embodiments, for example when used in consumer products such as a detergent, a fabric conditioner, a hand dishwash, or a home cleaner, the malodor-counteracting compound described herein may be present in an amount ranging from about 0.01% wt to about 10% wt. In some embodiments, for example when used in consumer products such as a detergent, a fabric conditioner, a hand dishwash, or a home cleaner, the malodor-counteracting compound described herein may be present in an amount ranging from about 2% wt to about 8% wt. In some embodiments, for example when used in consumer products such as a detergent, a fabric conditioner, a hand dishwash, or a home cleaner, the malodor-counteracting compound described herein may be present in an amount of about 5.0% wt.

In some embodiments, for example when used in consumer products such as candles, enclosed air care (e.g., propellant-based sprays (acrosol sprays), water-based sprays (trigger sprays)), toilet blocks, or plastic articles (e.g., garbage bag, trashcan liner), the malodor-counteracting compound described herein may be present in an amount of about or at least 0.01, 0.5, 1, 10, 20, 30, 40, 50, 60, 70, 80, 90, or 100% wt.

In some embodiments, for example when used in consumer products to counteract gaseous malodor, the malodor-counteracting compound of the present invention may be present in an amount ranging from about 0.01 to 1 mg per cubic meter of air.

### II. METHODS OF COUNTERACTING MALODOR

Provided herein are methods of counteracting malodors including the use of a malodor-counteracting compound described herein (see, Section I-A), a malodor-counteracting composition described herein (see, Section I-B), or a consumer product containing a malodor-counteracting compound or a malodor-counteracting composition described herein (see, Section I-C). In some embodiments, the method includes counteracting a malodor present in an air space. Non-limiting examples of air spaces containing malodor to be counteracted include homes, offices, gyms, and rooms therein, e.g., bathrooms, kitchens, bedrooms, living rooms, locker rooms, workout rooms, garages, etc. In some embodiments, the method includes counteracting a malodor present on a substrate. In some embodiments, the substrate is a fabric. Non-limiting examples of fabric include clothing, furniture, curtains, drapes, wall hangings, carpets, rugs, etc. In some embodiments, the substrate is human tissue. In some embodiments, the human tissue is axillary, scalp, face, or foot skin.

In some embodiments, the malodor counteracted is a body malodor. In some embodiments, the body malodor is a sweat malodor. In some embodiments, the body malodor is an axillary malodor. In some embodiments, the body malodor is a scalp malodor. In some embodiments, the malodor counteracted is an environmental malodor. In some embodiments, the environmental malodor is a bathroom malodor, e.g., urine and/or excrement malodor. In some embodiments, the environmental malodor is a mildew malodor. In some embodiments, the environmental malodor is a mold malodor. In some embodiments, the environmental malodor is a pet malodor. In some embodiments, the environmental malodor is a smoke, e.g., cigarette, cigar, malodor.

In some embodiments, the method of counteracting the malodor includes introducing the malodor-counteracting compound, the malodor-counteracting composition, or the consumer product into an air space. In some embodiments, the method of counteracting the malodor includes introducing the malodor-counteracting compound, the malodor-counteracting composition, or the consumer product onto a substrate. It will be appreciated that different formulations and/or consumer products may be used to counteract malodors depending on how they present, e.g., in an air space or on a substrate.

### III. EXEMPLARY EMBODIMENTS

Among the provided embodiments are:
1. A method of counteracting sweat malodor in an air space or a substrate comprising the step of introducing a composition into the air space or the substrate, wherein the composition comprises 2,2′-(octylimino) bis [ethanol].
2. The method of embodiment 1, wherein the composition further comprises a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, undecavertol, and a mixture thereof.
3. The method of embodiment 2, wherein 2,2′-(octylimino) bis [ethanol] and the fragrance compound have a weight ratio of at least 1:1.
4. The method of embodiment 3, wherein the weight ratio is from 2:1 to 4:1.
5. A composition for counteracting sweat malodor in air space or a substrate comprising 2,2′-(octylimino)bis[ethanol].
6. The composition of embodiment 5 further comprising a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, undecavertol and a mixture thereof.
7. A functional product comprising a composition for counteracting sweat malodor in air space or a substrate, wherein the composition comprises 2,2′-(octylimino) bis [ethanol].
8. The functional product of embodiment 7, wherein the function product is selected from the group consisting of a room freshener spray, a fragrance diffuser, a candle, a sachet, a clothes deodorant, a detergent, a fabric softener, a fabric refresher, a linen spray, a disposable diaper, a diaper pail deodorant, an antiperspirant, a deodorant, a garbage bag, a car freshener, a pet care product, and an animal litter material.
9. The functional product of embodiment 7, wherein the composition further comprises a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, undecavertol, and a mixture thereof.
10. The fragrance composition of embodiment 5 further comprising a polymer.
11. The fragrance formulation of embodiment 10, wherein the polymer is selected from the group consisting of polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde) and a combination thereof.

### IV. EXAMPLES

The following examples are included for illustrative purposes only and are not intended to limit the scope of the invention.

### Example 1: Preparation of Test Samples

A series of test solutions comprising (i) 2,2′-(octylimino)bis[ethanol] (“Octyl”) or an analog thereof, methyldiethanolamine (“Methyl”) (CAS 105-59-9) (commercially available from Alfa Aesar Materials Company) or Rewoquat WE 28 E (“Rewo”) (commercially available from Evonik Industries); and (ii) a fragrance compound selected from the group consisting of phenethyl alcohol, tetrahydrolinalool, linalool, 3-phenylpropanal, cinnamaldehyde, decanal, ethyl linalool, dihydromyrcenol, 2-methyldecanal, Veridian, and undecavertol containing a low level of Veridian were prepared in ethanol, wherein 2,2′-(octylimino)bis[ethanol] or an analog thereof was available at a concentration of 14% and the fragrance compound was available at a concentration of 7%.

A series of control solutions containing each of the above fragrance compounds were similarly prepare in ethanol at a concentration of 7%.

### Example 2: Improvement of Fragrance Retention

Fragrance retention was evaluated by the quantitative determination of the fragrance compounds in space, which was conducted using headspace technique, total ion count mass spectrometry.

Testing Procedure: The test samples of EXAMPLE 1 (10 mL) were each deposited onto a 1″ blotter paper strip resting in a precleaned 20 mL vial for volatile organic compounds (VOC) sampling. Three replicate samples were prepared for each test sample including the control solutions. Vials were then stored at 37° C. in a ventilated oven for 3-6 hours. The vials were capped and equilibrated to room temperature. Headspace was collected onto Gerstel Tenax-TA tubes, desorbed using the GERSTEL Thermal Desorption Unit (TDU), and analyzed with gas chromatography-mass spectrometry (GC-MS).

Results and Discussion: Average of total ion count (TIC) and standard deviation (SD) of the test samples are reported in the following. The TIC ratios of 2,2′-(octylimino)bis[ethanol] (“Octyl”), methyldiethanolamine (“Methyl”) and Rewoquat® WE 28 E (“Rewo”) test solutions and the control solutions are represented as RO, RM and RR, respectively.

TABLE E1

| | | Headspace Intensity (×1000) (TIC ± SD) | | | | | | |
|---|---|---|---|---|---|---|---|---|
| | Fragrance | Control | Octyl | RO | Methyl | RM | Rewo | RR |
| 1 | Phenethyl Alcohol | 4753 ± | 30205 ± | 6.35 | 23673 ± | 4.98 | 20202 ± | 4.25 |
| | | 1140 | 3363 | | 934 | | 1784 | |
| 2 | Tetrahydrolinalool | 2142 ± | 31294 ± | 14.61 | 4560 ± | 2.13 | 5814 ± | 2.71 |
| | | 802 | 7532 | | 308 | | 46100 | |
| 3 | Linalool | 4825 ± | 58848 ± | 12.2 | 11615 ± | 2.41 | 15232 ± | 3.16 |
| | | 3168 | 25517 | | 2173 | | 11760 | |
| 4 | 3-Phenylpropanal | 3744 ± | 15488 ± | 4.14 | 7094 ± | 1.89 | 9260 ± | 2.47 |
| | | 1745 | 3359 | | 1077 | | 3123 | |
| 5 | Cinnamaldehyde | 4554 ± | 25545 ± | 5.61 | 12429 ± | 2.73 | 16335 ± | 3.59 |
| | | 450 | 3065 | | 3409 | | 222 | |
| 6 | Decanal | 915 ± | 5654 ± | 6.18 | 1338 ± | 1.46 | 1138 ± | 1.24 |
| | | 63 | 3657 | | 375 | | 283 | |
| 7 | Ethyl Linalool | 1433 ± | 22231 ± | 15.51 | 7994 ± | 5.58 | 5153 ± | 3.6 |
| | | 610 | 4981 | | 2065 | | 4200 | |
| 8 | Dihydromyrcenol | 5105 ± | 61187 ± | 11.99 | 11518 ± | 2.26 | 11966 ± | 2.34 |
| | | 1071 | 5955 | | 4355 | | 8256 | |
| 9 | 2-Methyldecanal | 490 ± | 13681 ± | 27.92 | 1517 ± | 3.1 | 1869 ± | 3.82 |
| | | 104 | 4752 | | 212 | | 714 | |
| 10 | Veridian | 1426 ± | 5167 ± | 3.62 | 2360 ± | 1.65 | 1145 ± | 0.8 |
| | | 257 | 803 | | 723 | | 563 | |
| 11 | Undecavertol | 236 ± | 2827 ± | 11.97 | 863 ± | 3.65 | 765 ± | 3.24 |
| | | 97 | 1512 | | 214 | | 498 | |

Among all the test samples, 2,2′-(octylimino)bis[ethanol] provided higher TIC when compared with methyldiethanolamine and Rewoquat® WE 28 E. In particular, 2,2′-(octylimino)bis[ethanol] exhibited superior performance when combined with tetrahydrolinalool, linalool, decanal, ethyl linalool, dihydromyrcenol, 2-methyldecanal or undecavertol.

### Example 3: Unexpected Selectivity of 2,2′-(Octylimino)bis[ethanol] Compositions

The improvement of fragrance retention provided by 2,2′-(octylimino)bis[ethanol] is surprising and unexpected because such an effect depends on selective fragrance compounds. 2,2′-(Octylimino)bis[ethanol] does not provide prolonged retention to all fragrance compounds.

For example, 2,2′-(octylimino)bis[ethanol] failed to achieve prolonged fragrance retention when combined with Delphone (CAS No. 4819-67-4), Verdox (CAS No. 88-41-5), Floriffol (CAS No. 185019-16-3), Allyl Amyl Glycolate (CAS No. 124899-75-8), dimethyl benzyl carbinyl acetate (CAS No. 151-05-3), Delta Damascone (CAS No. 57378-68-4), Styralyl acetate (CAS No. 93-92-5), dimethyl octanol (CAS No. 106-21-8), fleuramone (CAS No. 137-03-1), geranyl acetate (CAS No. 105-87-3), Aupebine (CAS No. 123-11-5), Citronellol 950 (CAS No. 106-22-9), alpha-isomethyl ionone (CAS No. 127-51-5), Cashmeran (CAS No. 33704-61-9), Geraniol 980 Pure (CAS No. 106-24-1), beta-ionon (CAS No. 14901-07-6), Amber Xtreme and Calone (CAS No. 28940-11-6).

### Example 4: Evaluation of 2,2′-(Octylimino)bis[ethanol] and Fragrance Compound Combinations at Different Ratios

A series of solutions containing 2,2′-(octylimino)bis[ethanol] (“Octyl”) and the fragrance compound dihydromyrcenol (“DHM”) at different ratios were prepared similarly according to EXAMPLE 1 and evaluated for fragrance retention properties using the TIC mass spectrometry. The average of TIC and SD as well as the TIC ratios of the test and control samples (RO) are reported in the following.

TABLE E2

| Ratio (Octyl:DHM) | Headspace Intensity (x1000) (TIC ± SD) | RO |
|---|---|---|
| Control | 3674 ± 855 | N/A |
| 1:4 | 3548 ± 352 | 0.97 |
| 1:2 | 4568 ± 731 | 1.24 |
| 1:1 | 11823 ± 2192 | 3.22 |
| 2:1 | 51514 ± 15526 | 14.02 |
| 4:1 | 64099 ± 5535 | 17.45 |
| 5:1 | 49089 ± 3127 | 13.36 |
| 6:1 | 41558 ± 5357 | 11.31 |
| 7:1 | 40645 ± 2927 | 11.06 |

It was surprisingly found that only combinations of 2,2′-(octylimino)bis[ethanol] and dihydromyrcenol having a weight ratio of at least 1:1 and preferably from 2:1 to 4:1 exhibited superior performance.

### Example 5: Assessment of Malodor Counteracting Behavior

Establishment of Malodor Models: The sweat, bathroom, mildew, and smoke malodor models were prepared based on Applicants' proprietary formulations for assessing the effectiveness of various malodor counteractants.

Preparation of Test Samples: Samples of mixtures of a malodor material and (i) various test compounds including 2,2′-(octylimino)bis[ethanol] (“Octyl”), methyldiethanolamine (“Methyl”), 2,2′-(butylimino)bis[ethanol] (“Butyl”) (CAS No. 102-79-4), 2,2′-(dodecylimino)bis[ethanol] (“Dodecyl”) (CAS No. 1541-67-9) and 2,2′-(octadecylimino)bis[ethanol] (“Octadecyl”) (CAS No. 10213-78-2) diluted in a solvent (0.5%), respectively; or (ii) a solvent alone control were pipetted into plastic pellets and placed in plastic squeeze bottles. The bottles were capped and the samples were allowed to equilibrate for one hour before the testing.

Testing Procedure: 24 Trained panelists (consisting of men/women with an age range of 25 to 55). The panelists were instructed to take the steps of i) uncap a bottle; ii) place their noses at a distance of about 3-4 inches above the opening; iii) take short sniffs for 3 seconds while squeezing the bottle; and v) enter a rating of overall intensity and malodor intensity on a handheld computer.

The overall and malodor intensity was rated using the Labeled Magnitude Scale (LMS) [Green, et al., Chemical Senses, 21(3), June 1996, 323-334]. Percent malodor reduction (“% MOR”) represents the perceived reduction in mean malodor intensity of the sample containing the malodor in the presence of a malodor counteractant relative to the negative control (Malodor Alone).

Results and Discussion: The mean ranks of the malodor coverage for the above test were as follows:

TABLE E3

| | % MOR | | | | |
|---|---|---|---|---|---|
| | Octyl | Methyl | Butyl | Dodecyl | Octadecyl |
| Sweat | 66 | 55 | 48 | 51 | 32 |
| Bathroom | 21 | 10 | 27 | 21 | 9 |
| Mildew | 17 | 14 | 5 | 5 | 7 |
| Smoke | 6 | 4 | 7 | 2 | 0 |

When compared with its analogs, 2,2′-(Octylimino)bis[ethanol] of the present invention was confirmed to be significantly and specifically effective in counteracting sweat malodor.

### Example 6: Evaluation of 2,2′-(alkylimino)bis[ethanol]s Having Different Alkyl Chain Lengths on Aldehyde Stability

To assess the impact of 2,2′-(alkylimino)bis[ethanol] s having different alkyl chain lengths on the stability of aldehydes, a fragrance accord containing equal proportions by weight of octanal, nonanal, decanal, 10-undecenal, and dodecanal was prepared. Individual test samples were prepared by mixing the fragrance accord with one 2,2′-(alkylimino)bis[ethanol] having either a C₄, C₈, C₁₂, or C₁₈ alkyl chain length at a weight ratio of 1:2 (2,2′-(alkylimino)bis[ethanol]:fragrance accord). Control samples contained the fragrance ingredient without any 2,2′-(alkylimino)bis[ethanol] s. All samples were prepared in triplicate and stored at 40° C. for 4 weeks. No efforts were made to exclude air from the samples. Immediately prior to analysis by GC-MS, samples were diluted with acetone to give 1% w/w fragrance accord. GC peak areas are reported as total ion count (TIC) in TABLE E4 and the difference relative to control is shown in TABLE E5. The 2,2′-(alkylimino)bis[ethanol] tested is denoted by the alkyl chain length (C4, C8, C12, C18) and the control is denoted by “Control” in the tables.

TABLE E4
TIC (mean ± standard deviation) for each test and control sample.

| | Control | C4 | C8 | C12 | C18 |
|---|---|---|---|---|---|
| Octanal | 7.55E+07 ± | 2.70E+07 ± | 2.66E+07 ± | 2.39E+07 ± | 2.05E+07 ± |
| | 7.27E+06 | 2.08E+05 | 2.38E+06 | 1.32E+06 | 9.57E+05 |
| Nonanal | 8.65E+07 ± | 3.34E+07 ± | 3.26E+07 ± | 2.98E+07 ± | 2.55E+07 ± |
| | 9.65E+06 | 4.55E+05 | 3.21E+06 | 1.63E+06 | 1.15E+06 |
| Decanal | 9.02E+07 ± | 3.85E+07 ± | 3.80E+07 ± | 3.52E+07 ± | 2.99E+07 ± |
| | 1.06E+07 | 5.64E+05 | 3.76E+06 | 1.86E+06 | 1.18E+06 |
| 10-undecanal | 9.90E+07 ± | 4.59E+07 ± | 4.52E+07 ± | 4.15E+07 ± | 3.51E+07 ± |
| (Ulenic) | 1.24E+07 | 7.71E+05 | 4.23E+06 | 1.40E+06 | 1.15E+06 |
| Dodecanal (Lauric) | 9.62E+07 ± | 5.36E+07 ± | 5.08E+07 ± | 4.62E+07 ± | 3.85E+07 ± |
| | 1.20E+07 | 2.89E+05 | 4.82E+06 | 1.15E+06 | 1.18E+06 |

TABLE E5
Percent change in TIC relative to control.

| | C4 | C8 | C12 | C18 |
|---|---|---|---|---|
| Octanal | −64% | −65% | −68% | −73% |
| Nonanal | −61% | −62% | −66% | −71% |
| Decanal | −57% | −58% | −61% | −67% |
| 10-undecanal (Ulenic) | −54% | −54% | −58% | −65% |
| Dodecanal (Lauric) | −44% | −47% | −52% | −60% |

These results suggest, unexpectedly, that 2,2′-(alkylimino)bis[ethanol] s having shorter alkyl chain lengths have less impact on aldehyde stability compared to those with longer chain lengths. These results are supportive of the use of 2,2′-(alkylimino)bis[ethanol] having shorter alkyl chain lengths (e.g., C₄₋₁₂) in fragrance compositions containing aldehydes.

### Example 7: Evaluation of 2,2′-(alkylimino)bis[ethanol]s Having Different Alkyl Chain Lengths on the Stability of Ester Fragrance Ingredients

To assess the impact of 2,2′-(alkylimino)bis[ethanol] s having different alkyl chain lengths on the stability of ester fragrance ingredients, three fragrance accords were created. The first fragrance accord contained equal proportions by weight of cis-3-hexenyl acetate, benzyl acetate, geranyl acetate, methyl dihydrojasmonate, hexyl salicylate, (E)-oxacycloheptadec-10-en-2-one (ambrettolide), and ethylene brassylate was prepared. The second fragrance accord contained equal proportions by weight of prenyl acetate, allyl caproate, allyl heptanoate, allyl amyl glycolate, linalyl acetate, allyl caprylate, pinocarvyl acetate, ethyl linalyl acetate, neryl acetate, geranyl acetate, linalyl isobutyrate, cyclogalbanate, allyl cyclohexyl propionate, dihydrocuminyl acetate, cinnamyl acetate, allyl phenoxyacetate, geranyl propionate, geranyl isobutyrate, geranyl tiglate, and farnesyl acetate. The third fragrance accord contained equal proportions by weight of p-cresyl acetate, methyl salicylate, ethyl salicylate, Honey F, p-cresyl isobutyrate, methyl anisate, coumarin, isobutyl salicylate, maltol isobutyrate, cugenyl acetate, amyl salicylate, Celeriax, Oceanol, Iso Butavan, cis-3-hexenyl salicylate, hexyl salicylate, Veramoss, cyclohexyl salicylate, and phenethyl salicylate. In the third fragrance accord, the amyl salicylate was a mixture of 2-methylbutyl and n-amyl esters in approximately a 1:2 ratio.

Individual test samples were prepared by mixing each of the fragrance accords with one 2,2′-(alkylimino)bis[ethanol] having either a C₁, C₄, C₈, or C₁₂ alkyl chain length at a weight ratio of 1:2 (2,2′-(alkylimino)bis[ethanol]:fragrance accord). Control samples contained the fragrance accord without any 2,2′-(alkylimino)bis[ethanol]s. All samples were prepared in triplicate and stored at 40° C. for 4 weeks. No efforts were made to exclude air from the samples. Immediately prior to analysis by GC-MS, samples were diluted with acetone to give 1% w/w fragrance accord. GC peak areas are reported as total ion count (TIC) in TABLE E6 and the difference relative to control is shown in TABLE E7. In the second fragrance accord, it should be appreciated that geranyl propionate contains about 25% of the corresponding citronellyl ester, thus the additional peak is noted in TABLES E6-E7. The 2,2′-(alkylimino)bis[ethanol] tested is denoted by the alkyl chain length (C1, C4, C8, C12) and the control is denoted by “Control” in the tables.

TABLE E6
TIC (mean ± standard deviation) for test and control samples of each
fragrance accord. Isomer peaks for an ingredient are denoted by “i” and “ii”.

| | | | | |
|---|---|---|---|---|
| Control | C1 | C4 | C8 | C12 |

FIRST FRAGRANCE ACCORD

| | | | | | |
|---|---|---|---|---|---|
| (E)- | 8.49E+07 ± | 7.02E+07 ± | 7.73E+07 ± | 8.11E+07 ± | 7.98E+07 ± |
| oxacycloheptadec- | 3.82E+06 | 5.59E+06 | 6.86E+06 | 3.77E+06 | 1.62E+06 |
| 10-en-2-one | | | | | |
| Benz Acet | 8.33E+07 ± | 2.33E+07 ± | 3.97E+07 ± | 5.58E+07 ± | 6.25E+07 ± |
| | 4.33E+06 | 1.93E+06 | 3.83E+06 | 2.23E+06 | 1.05E+06 |
| Cis-3-Hexenyl Acet | 6.60E+07 ± | 4.19E+07 ± | 5.22E+07 ± | 5.97E+07 ± | 6.25E+07 ± |
| | 3.20E+06 | 3.76E+06 | 4.97E+06 | 2.58E+06 | 1.28E+06 |
| Ethylene Brassylate | 8.30E+07 ± | 3.44E+07 ± | 5.26E+07 ± | 6.48E+07 ± | 7.02E+07 ± |
| | 4.00E+06 | 3.00E+06 | 4.77E+06 | 3.09E+06 | 1.80E+06 |
| Geranyl Acet | 8.08E+07 ± | 4.98E+07 ± | 6.10E+07 ± | 7.08E+07 ± | 7.36E+07 ± |
| | 4.26E+06 | 4.35E+06 | 5.99E+06 | 3.39E+06 | 1.89E+06 |
| Hexyl Sal | 8.84E+07 ± | 4.11E+07 ± | 6.13E+07 ± | 7.23E+07 ± | 7.56E+07 ± |
| | 5.10E+06 | 3.80E+06 | 5.88E+06 | 3.84E+06 | 2.10E+06 |
| Meth DH Jasmonate i | 7.38E+07 ± | 4.83E+07 ± | 5.91E+07 ± | 6.67E+07 ± | 6.76E+07 ± |
| | 3.71E+06 | 4.10E+06 | 5.51E+06 | 3.00E+06 | 1.42E+06 |
| Meth DH Jasmonate | 7.94E+06 ± | 4.13E+06 ± | 5.34E+06 ± | 6.13E+06 ± | 6.27E+06 ± |
| ii | 3.52E+05 | 3.46E+05 | 4.77E+05 | 2.48E+05 | 1.22E+05 |

SECOND FRAGRANCE ACCORD

| | | | | | |
|---|---|---|---|---|---|
| Allyl Amyl Glycolate | 2.34E+07 ± | 6.57E+06 ± | 5.73E+06 ± | 6.21E+06 ± | 7.62E+06 ± |
| | 1.12E+06 | 2.98E+05 | 3.60E+05 | 5.54E+05 | 8.50E+05 |
| Allyl Caproate | 2.31E+07 ± | 1.61E+07 ± | 1.87E+07 ± | 2.10E+07 ± | 2.14E+07 ± |
| | 1.08E+06 | 8.44E+05 | 1.40E+06 | 1.31E+06 | 2.66E+06 |
| Allyl Caprylate | 2.60E+07 ± | 1.87E+07 ± | 2.10E+07 ± | 2.32E+07 ± | 2.37E+07 ± |
| | 1.16E+06 | 8.94E+05 | 1.51E+06 | 1.53E+06 | 2.74E+06 |
| Citronellyl Prop | 8.36E+06 ± | 8.27E+06 ± | 7.94E+06 ± | 7.92E+06 ± | 7.80E+06 ± |
| | 3.52E+05 | 4.35E+05 | 7.54E+05 | 5.23E+05 | 8.75E+05 |
| Cyclogalbanate | 2.68E+07 ± | 3.00E+06 ± | 3.95E+06 ± | 6.02E+06 ± | 7.93E+06 ± |
| | 1.31E+06 | 1.14E+05 | 2.79E+05 | 6.48E+05 | 8.67E+05 |
| Eth Linalyl Acet i | 1.30E+07 ± | 1.43E+07 ± | 1.22E+07 ± | 1.26E+07 ± | 1.26E+07 ± |
| | 6.59E+05 | 6.15E+05 | 9.84E+05 | 8.55E+05 | 1.50E+06 |
| Eth Linalyl Acet ii | 1.98E+07 ± | 1.88E+07 ± | 1.86E+07 ± | 1.93E+07 ± | 1.92E+07 ± |
| | 9.84E+05 | 9.85E+05 | 1.16E+06 | 1.27E+06 | 2.35E+06 |
| Farnesyl Acet i | 9.25E+06 ± | 6.01E+06 ± | 7.15E+06 ± | 7.53E+06 ± | 7.95E+06 ± |
| | 4.58E+05 | 2.99E+05 | 4.92E+05 | 5.74E+05 | 9.69E+05 |
| Farnesyl Acet ii | 1.50E+07 ± | 9.66E+06 ± | 1.16E+07 ± | 1.31E+07 ± | 1.37E+07 ± |
| | 7.79E+05 | 5.24E+05 | 7.91E+05 | 1.00E+06 | 1.48E+06 |
| Geranyl Acet | 4.24E+07 ± | 2.52E+07 ± | 3.13E+07 ± | 3.57E+07 ± | 3.69E+07 ± |
| | 2.03E+06 | 1.28E+06 | 2.28E+06 | 2.75E+06 | 4.72E+06 |
| Geranyl Iso Buty | 1.77E+07 ± | 1.57E+07 ± | 1.62E+07 ± | 1.68E+07 ± | 1.67E+07 ± |
| | 8.84E+05 | 7.54E+05 | 1.06E+06 | 1.17E+06 | 2.01E+06 |
| Geranyl Prop | 1.63E+07 ± | 1.21E+07 ± | 1.36E+07 ± | 1.47E+07 ± | 1.49E+07 ± |
| | 8.18E+05 | 6.00E+05 | 8.71E+05 | 1.04E+06 | 1.73E+06 |
| Geranyl Tiglate | 3.06E+07 ± | 2.84E+07 ± | 2.86E+07 ± | 2.93E+07 ± | 2.90E+07 ± |
| | 1.66E+06 | 1.58E+06 | 1.71E+06 | 2.22E+06 | 3.36E+06 |
| Linalyl Acet | 3.26E+07 ± | 3.10E+07 ± | 3.04E+07 ± | 3.19E+07 ± | 3.18E+07 ± |
| | 1.67E+06 | 1.59E+06 | 1.97E+06 | 2.03E+06 | 3.90E+06 |
| Linalyl Iso Buty | 3.17E+07 ± | 3.17E+07 ± | 3.09E+07 ± | 3.15E+07 ± | 3.11E+07 ± |
| | 1.71E+06 | 1.68E+06 | 1.67E+06 | 2.18E+06 | 3.58E+06 |
| Neryl Acet | 1.80E+07 ± | 1.17E+07 ± | 1.40E+07 ± | 1.56E+07 ± | 1.61E+07 ± |
| | 8.68E+05 | 5.76E+05 | 9.77E+05 | 1.09E+06 | 1.88E+06 |
| Pinocarvyl Acet | 3.07E+07 ± | 2.82E+07 ± | 2.83E+07 ± | 2.98E+07 ± | 2.97E+07 ± |
| | 1.40E+06 | 1.29E+06 | 1.84E+06 | 1.81E+06 | 3.37E+06 |
| Prenyl Acet | 2.17E+07 ± | 1.28E+07 ± | 1.62E+07 ± | 1.90E+07 ± | 1.97E+07 ± |
| | 1.11E+06 | 7.59E+05 | 1.24E+06 | 1.31E+06 | 2.60E+06 |

THIRD FRAGRANCE ACCORD

| | | | | | |
|---|---|---|---|---|---|
| Amyl Sal | 2.24E+07 ± | 1.10E+07 ± | 1.57E+07 ± | 1.85E+07 ± | 2.07E+07 ± |
| | 7.90E+05 | 5.86E+04 | 6.12E+05 | 9.08E+05 | 1.71E+06 |
| Celeriax i | 2.65E+07 ± | Not detected | Not | 6.17E+05 ± | 3.68E+06 ± |
| | 8.10E+05 | | detected | 1.07E+06 | 2.72E+05 |
| Celeriax ii | 2.08E+06 ± | Not detected | Not | Not detected | Not |
| | 6.55E+04 | | detected | | detected |
| Cis-3-Hexenyl Sal | 2.98E+07 ± | 1.10E+07 ± | 1.72E+07 ± | 2.24E+07 ± | 2.61E+07 ± |
| | 9.01E+05 | 1.40E+05 | 7.08E+05 | 1.11E+06 | 2.17E+06 |
| Coumarin | 2.61E+07 ± | 2.43E+07 ± | 2.46E+07 ± | 2.47E+07 ± | 2.56E+07 ± |
| | 9.77E+05 | 4.94E+05 | 8.76E+05 | 1.22E+06 | 1.94E+06 |
| Cresyl Acet | 3.28E+07 ± | Not detected | Not | Not detected | Not |
| | 9.49E+05 | | detected | | detected |
| Cresyl Iso Buty | 3.32E+07 ± | Not detected | Not | Not detected | Not |
| | 8.63E+05 | | detected | | detected |
| Cyclohexyl Sal | 3.33E+07 ± | 2.92E+07 ± | 3.17E+07 ± | 3.13E+07 ± | 3.24E+07 ± |
| | 1.09E+06 | 6.34E+05 | 1.14E+06 | 1.58E+06 | 2.57E+06 |
| Eth Sal | 3.40E+07 ± | 1.36E+07 ± | 2.10E+07 ± | 2.65E+07 ± | 3.04E+07 ± |
| | 1.03E+06 | 2.56E+05 | 8.36E+05 | 1.39E+06 | 2.09E+06 |
| Hexyl Sal | 3.36E+07 ± | 1.73E+07 ± | 2.39E+07 ± | 2.78E+07 ± | 3.08E+07 ± |
| | 1.07E+06 | 2.26E+05 | 8.92E+05 | 1.32E+06 | 2.51E+06 |
| Honey F i | 8.08E+06 ± | Not detected | Not | Not detected | Not |
| | 2.29E+05 | | detected | | detected |
| Honey F ii | 2.17E+07 ± | Not detected | Not | Not detected | Not |
| | 6.41E+05 | | detected | | detected |
| Iso Amyl Sal | 1.15E+07 ± | 8.66E+06 ± | 1.14E+07 ± | 1.22E+07 ± | 1.26E+07 ± |
| | 3.50E+05 | 1.51E+05 | 4.24E+05 | 5.76E+05 | 9.22E+05 |
| Iso Butavan | 2.38E+07 ± | Not detected | Not | Not detected | Not |
| | 7.31E+05 | | detected | | detected |
| Maltol Iso Buty | 1.83E+07 ± | Not detected | Not | Not detected | 1.83E+06 ± |
| | 7.05E+05 | | detected | | 2.18E+05 |
| Meth Anisate | 3.28E+07 ± | 2.72E+07 ± | 2.94E+07 ± | 3.04E+07 ± | 3.19E+07 ± |
| | 1.07E+06 | 4.76E+05 | 1.05E+06 | 1.38E+06 | 2.20E+06 |
| Meth Sal | 3.05E+07 ± | 1.07E+07 ± | 1.05E+07 ± | 1.52E+07 ± | 2.12E+07 ± |
| | 9.79E+05 | 2.32E+05 | 4.12E+05 | 7.91E+05 | 1.52E+06 |
| Oceanol | 2.38E+07 ± | 2.06E+07 ± | 2.22E+07 ± | 2.25E+07 ± | 2.37E+07 ± |
| | 7.85E+05 | 4.71E+05 | 8.53E+05 | 1.65E+06 | 2.48E+06 |

TABLE E7
Percent change in TIC relative to control for test samples
of each fragrance accord.

| | | | |
|---|---|---|---|
| C1 | C4 | C8 | C12 |

FIRST FRAGRANCE ACCORD

| | | | | |
|---|---|---|---|---|
| (E)-oxacycloheptadec-10-en- | −17% | −9% | −4% | −6% |
| 2-one | | | | |
| Benz Acet | −72% | −52% | −33% | −25% |
| Cis-3-Hexenyl Acet | −36% | −21% | −9% | −5% |
| Ethylene Brassylate | −59% | −37% | −22% | −15% |
| Geranyl Acet | −38% | −25% | −12% | −9% |
| Hexyl Sal | −53% | −31% | −18% | −14% |
| Meth DH Jasmonate i | −35% | −20% | −10% | −8% |
| Meth DH Jasmonate ii | −48% | −33% | −23% | −21% |

SECOND FRAGRANCE ACCORD

| | | | | |
|---|---|---|---|---|
| Allyl Amyl Glycolate | −72% | −76% | −74% | −67% |
| Allyl Caproate | −31% | −19% | −9% | −8% |
| Allyl Caprylate | −28% | −19% | −11% | −9% |
| Citronellyl Prop | −1% | −5% | −5% | −7% |
| Cyclogalbanate | −89% | −85% | −78% | −70% |
| Eth Linalyl Acet i | 10% | −6% | −3% | −3% |
| Eth Linalyl Acet ii | −5% | −6% | −3% | −3% |
| Farnesyl Acet i | −35% | −23% | −19% | −14% |
| Farnesyl Acet ii | −36% | −23% | −13% | −8% |
| Geranyl Acet | −41% | −26% | −16% | −13% |
| Geranyl Iso Buty | −11% | −8% | −5% | −5% |
| Geranyl Prop | −26% | −16% | −10% | −9% |
| Geranyl Tiglate | −7% | −7% | −4% | −5% |
| Linalyl Acet | −5% | −7% | −2% | −2% |
| Linalyl Iso Buty | 0% | −3% | 0% | −2% |
| Neryl Acet | −35% | −22% | −13% | −11% |
| Pinocarvyl Acet | −8% | −8% | −3% | −3% |
| Prenyl Acet | −41% | −25% | −12% | −9% |

THIRD FRAGRANCE ACCORD

| | | | | |
|---|---|---|---|---|
| Amyl Sal | −51% | −30% | −17% | −8% |
| Celeriax i | −100% | −100% | −98% | −86% |
| Celeriax ii | −100% | −100% | −100% | −100% |
| Cis-3-Hexenyl Sal | −63% | −42% | −25% | −13% |
| Coumarin | −7% | −6% | −5% | −2% |
| Cresyl Acet | −100% | −100% | −100% | −100% |
| Cresyl Iso Buty | −100% | −100% | −100% | −100% |
| Cyclohexyl Sal | −12% | −5% | −6% | −3% |
| Eth Sal | −60% | −38% | −22% | −11% |
| Hexyl Sal | −49% | −29% | −17% | −8% |
| Honey F i | −100% | −100% | −100% | −100% |
| Honey F ii | −100% | −100% | −100% | −100% |
| Iso Amyl Sal | −25% | −1% | 6% | 9% |
| Iso Butavan | −100% | −100% | −100% | −100% |
| Maltol Iso Buty | −100% | −100% | −100% | −90% |
| Meth Anisate | −17% | −10% | −7% | −3% |
| Meth Sal | −65% | −66% | −50% | −31% |
| Oceanol | −14% | −7% | −6% | 0% |

Stability of the dihydrocuminyl acetate and cinnamyl acetate present in the second fragrance accord was evaluated by detecting alcohol release by GC-MS. Results are shown in TABLE E8 below. The 2,2′-(alkylimino)bis[ethanol] tested is denoted by the alkyl chain length (C1, C4, C8, C12) and control is denoted by “Control” in the table.

TABLE E8
Alcohol release (mean ± standard deviation) measure by GC-MS for test
and control samples of the second fragrance accord.

| | Control | C1 | C4 | C8 | C12 |
|---|---|---|---|---|---|
| Cinnamyl Alc | Not detected | 1.18E+07 ± | 7.51E+06 ± | 5.13E+06 ± | 3.70E+06 ± |
| | | 6.82E+05 | 2.91E+05 | 2.40E+05 | 6.17E+05 |
| Dihydro Cuminyl Alc | Not detected | 7.77E+06 ± | 4.34E+06 ± | 2.78E+06 ± | 1.95E+06 ± |
| | | 3.78E+05 | 1.70E+05 | 5.51E+04 | 2.81E+05 |

These results suggest that the stability of esters is affected at least in part by the 2,2′-(alkylimino)bis[ethanol] alkyl chain length, with certain lengths having more preferable stability outcomes for certain esters. These results are supportive of the use of 2,2′-(alkylimino)bis[ethanol] having specific alkyl chain lengths (e.g., C₄₋₁₂) in fragrance compositions containing select ester fragrance ingredients.

### Example 8: Fabric Spray Application

The ability of 2,2′-(octylimino)bis[ethanol] (C8 linear alkyl) to affect the sensory perception of sweat malodor was evaluated in a fabric refresher spray application using a sweat malodor model based on a proprietary formulation.

Fabric refresher spray samples were prepared by adding and mixing 2,2′-(octylimino)bis[ethanol] or fragrance, Floral HCA (high coverage accord), to a fabric refresher spray base (see, TABLE E9 for base composition) in a pump spray glass container. Floral HCA is a proprietary accord of fragrance ingredients that (1) can be dosed at a low concentration while still providing a high level of sweat malodor coverage and (2) has demonstrated at least an 80% reduction of sweat malodor in prior sensory testing.

TABLE E9
Fabric refresher spray base composition.

| | Approximate % |
|---|---|
| | Concentration |
| INCI | in Base Formula |
| Aqua | 88-88.5 |
| Alcohol | 10.0-10.1 |
| PEG-40 Hydrogenated Castor Oil | 0.9-1.1 |
| Fragrance or 2,2′-(octylimino) | 0.1-2.0 |
| bis[ethanol] | |

Samples for evaluation were prepared by spraying 1 pump (˜0.18 g) of sweat malodor onto a cloth substrate (3.5 in diameter). After 1 minute, the fabric refresher spray test sample was used to spray 1 pump (0.12 g) of product onto the same cloth substrate. The cloth substrate was transferred to a 16 oz. jar and immediately closed with an air-tight lid. After 4 hours, samples were presented in a blind and random order to 6 trained panelists. The panelists were instructed to take the steps of i) uncap the jar; ii) place their noses at a distance of about 2-3 inches above the opening; iii) take short sniffs for 3 seconds; and iv) enter a rating of malodor intensity on a scale of 0 (lowest) to 10 (highest).

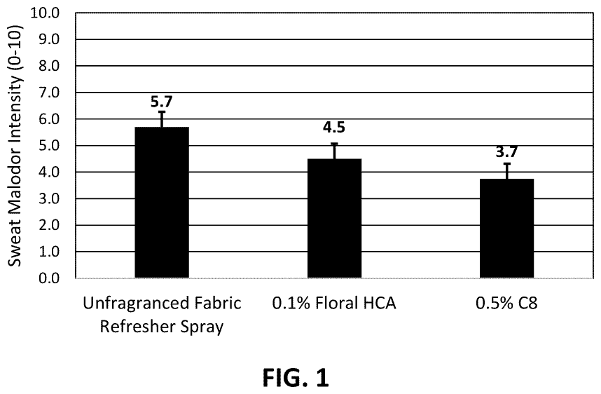

FIG. 1 is a bar chart showing the mean and standard deviation of the sweat malodor intensity rating provided by the panelists. As shown in FIG. 1, sweat malodor intensity ratings were lower when fabric spray including 2,2′-(octylimino)bis[ethanol] was used compared to sprays containing Flora HCA and unfragranced sprays.

These results are supportive of the ability of the 2,2′-(octylimino)bis[ethanol] to decrease sweat malodor intensity in cloth substrates.

### Example 9: Deodorant Application

The ability of 2,2′-(octylimino)bis[ethanol] to affect the sensory perception of sweat malodor was evaluated in-use with 33 subjects (19 men and 14 women between 20 and 50 years of age).

Test deodorants: Two aerosol candidates were prepared as follow: an exemplary non-antiperspirant (non-AP) spray and the exemplary non-AP spray including 0.5% 2,2′-(octylimino)bis[ethanol].

Experimental Procedure: For five days before the test sensory evaluation (i.e., the “test phase”), the subjects underwent a “wash-out phase” during which they substituted their normal bodywash with an unfragranced shower gel and were asked to not use any underarm product. On day 5 of the “wash-out phase,” subjects performed a workout session between 8 h and 12 h. The subjects were asked to rate the sweat malodor intensity of their axilla on a 0 (lowest) to 10 (highest) point scale before (at 8 h) and after (at T12 h) the workout session. The sensory results were used as a baseline.

On day 6 (“test phase”) subjects showered with the unfragranced shower gel and applied the 2 aerosol candidates (one to each axilla) through a standardized procedure with a blind and randomized (left/right) design. Each subject wore both candidates. The subjects performed a workout session between 8 h and 12 h. As with day 5 of the “wash-out phase,” the subjects rated the sweat malodor intensity of their axilla on a 0-10 point scale before (8 h) and after (12 h) the workout session.

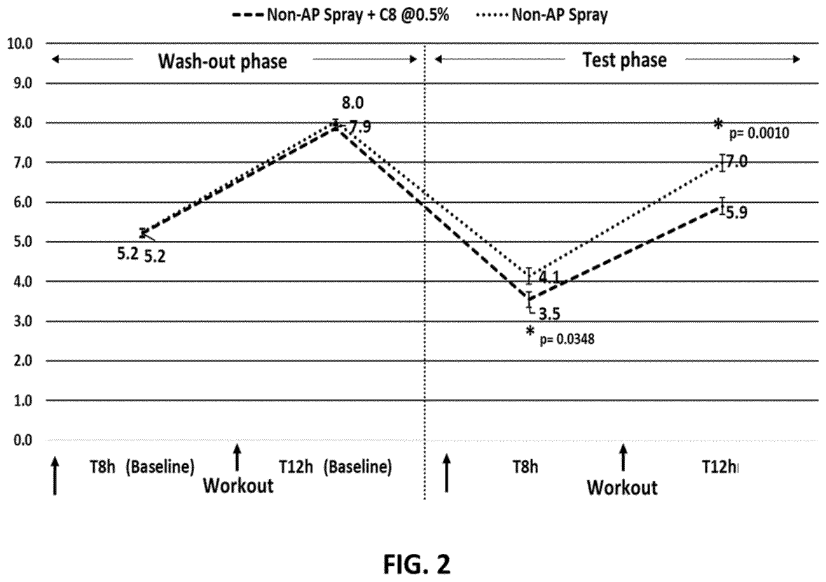

Results: FIG. 2 shows the mean and standard deviation of sweat malodor intensity ratings at 8 h and 12 h on day 5 of the “wash-out phase” and at 8 h and 12 h on day 6 (the “test phase”). As demonstrated in FIG. 2, sweat malodor intensity was reported as lower with the non-AP spray containing 2,2′-(octylimino)bis[ethanol] compared to the candidate lacking 2,2′-(octylimino)bis[ethanol] 8 h p=0.0348; 12 h p=0.0010).

These results suggest the ability of the 2,2′-(octylimino)bis[ethanol] to decrease the sweat malodor intensity when applied to human tissue.

### Example 10: Body Spray

A model body spray base was formulated to contain 1% isopropyl myristate and 48% ethanol (190 proof). An isovaleric acid malodor was prepared. A dilution of 2,2′-(octylimino)bis[ethanol] was prepared, 25% w/w in ethanol (190 proof). Concentrated hydrochloric acid was used as received (37% w/w in water). Test and control samples were prepared in triplicate in 20 mL VOC vials.

TABLE E10
Model body spray sample formulation.

| | Body | | 37% | Isovaleric |
|---|---|---|---|---|
| | Spray | 2,2′- | hydrochloric | acid |
| Sample | Base | (octylimino)bis[ethanol] | acid | malodor |
| Control | 960 mg | none | none | 40 mg |
| 0.5% w/w 2,2′- | 938 mg | 20 mg (25% w/w dilution) | 2.3 mg | 40 mg |
| (octylimino)bis[ethanol] | | | | |
| 2% w/w 2,2′- | 931 mg | 20 mg (undiluted) | 9.1 mg | 40 mg |
| (octylimino)bis[ethanol] | | | | |

Each sample was closed immediately with a cap containing PTFE faced 0.125″ silicone septum, and mixed. Each vial was analyzed by piercing with a needle and passing headspace through a Gerstel thermal desorption tube packed with Tenax-TA adsorbent, using a pump at 50 mL/min for 2 min. Tubes were desorbed on a thermal desorption unit connected to GC-MS for peak identification and quantification.

The results shown in TABLE E11 demonstrate that 2,2′-(octylimino)bis[ethanol] treated with stoichiometric amounts of hydrochloric acid were effective in lowering the headspace concentration of isovaleric acid relative to the control sample.

TABLE E11
Isovaleric acid (mean ± standard deviation) measure by
GC-MS for control and test samples.

| | Peak | | |
|---|---|---|---|
| | area, | | |
| | average | | Change |
| | of three | Standard | from |
| | replicates | deviation | control |
| Control | 22832.79 | 2448.481 | N/A |
| 0.5% 2,2′- | 5396 | 1061.631 | −76% |
| (octylimino)bis[ethanol] + | | | |
| HCl | | | |
| 2.0% 2,2′- | 1693 | 627.6469 | −93% |
| (octylimino)bis[ethanol] + | | | |
| HCl | | | |

These results support the ability of 2,2′-(octylimino)bis[ethanol] mixed with an acid to counteract malodor.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure. Although the invention may be described in connection with specific preferred embodiments, it should be understood that the invention as claimed should not be unduly limited to such specific embodiments. Indeed, various modifications of the described modes for carrying out the invention which are obvious to those skilled in chemistry, perfumery, or related fields are intended to be within the scope of the following claims.

1-2. (canceled)

3. A malodor-counteracting composition comprising a 2,2′-(alkylimino)bis[ethanol], wherein the alkyl is a C₄₋₁₂ alkyl, and one or more of:
(i) a solvent selected from the group consisting of IPM, DPG, triethyl citrate, and any combination thereof;
(ii) an antibacterial active selected from the group consisting of an antibacterial alcohol, a bactericidal acid, a diol, a polyol, a quaternary ammonium compound, silver metal, silver salt, and any combination thereof;
(iii) a water absorber selected from the group consisting of clay, aluminum salt, magnesium oxide, talc, polyacrylate, cellulose, magnesium sulfate, and any combination thereof;
(iv) a stabilizer selected from the group consisting of a UV filter, an antioxidant, a chelating agent, and any combination thereof; or
(v) an olfactory receptor blocker.

4. The malodor-counteracting composition of claim 3, further comprising one or more fragrance ingredients susceptible to hydrolysis, ethanolysis, solvolysis, retro-aldol elimination, dimerization, polymerization, and/or transesterification.

5. The malodor-counteracting composition of claim 4, wherein the one or more fragrance ingredients degrade by less than about 40%, optionally less than about 20%, in the presence of the 2,2′-(alkylimino)bis[ethanol], compared to the degradation of the one or more fragrance ingredients in a control condition lacking the 2,2′-(alkylimino)bis[ethanol].

6. The malodor-counteracting composition of claim 4, wherein the one or more fragrance ingredients comprise an aldehyde, an aldehyde precursor, an ester, an ester precursor, a lactone, a lactone precursor, or any combination thereof.

7. The malodor-counteracting composition of claim 3, wherein the 2,2′-(alkylimino)bis[ethanol] is in a protonated form, a neutral form, or a salt form.

8-9. (canceled)

10. The malodor-counteracting composition of claim 3, wherein the composition further comprises an acid selected from the group consisting of hydrochloric acid, citric acid, lactic acid, benzoic acid, ethylenediamine tetraacetic acid (EDTA), a plant-derived fatty-acid, and any combination thereof.

11. (canceled)

12. The malodor-counteracting composition of claim 3, wherein the alkyl is a C₈ linear alkyl.

13. The malodor-counteracting composition of claim 6, wherein the aldehyde has the formula: 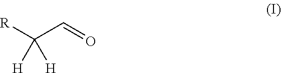
wherein
R is a C₁₋₁₀ alkyl, C₁₋₁₀ alkenyl, or C₁₋₁₀ alkynyl, optionally substituted by one or more of a hydroxyl, an ester, an ether, a cyclopentyl, a cyclohexyl, a cyclopentadienyl, a benzyl, or a furanyl group.

14. The malodor-counteracting composition of claim 6, wherein the aldehyde is octanal, nonanal, decanal, 10-undecenal, or dodecanal.

15-16. (canceled)

17. The malodor-counteracting composition of claim 6, wherein the ester is benzyl acetate, cis-3-hexenyl acetate, geranyl acetate, hexyl salicylate, cinnamyl acetate, (4-(prop-1-en-2-yl)cyclohex-1-en-1-yl)methyl acetate (dihydrocuminyl acetate), allyl 2-(cyclohexyloxy)acetate (Cyclogalbanate), 3-methylbut-2-en-1-yl acetate (prenyl acetate), anisyl acetate, benzyl butyrate, benzyl cinnamate, benzyl propionate, benzyl salicylate, 4-isopropylbenzyl acetate (cuminyl acetate), para-methylbenzyl acetate, amyl salicylate, cis-3-hexenyl salicylate, ethyl salicylate, methyl salicylate, methyl 2-(3-oxo-2-pentylcyclopentyl)acetate (methyl dihydrojasmonate), allyl caproate, allyl caprylate, farnesyl acetate, geranyl acetate, geranyl propionate, neryl acetate, benzyl benzoate, benzyl iso butyrate, or methyl 4-methoxybenzoate (methyl anisate).

18. The malodor-counteracting composition of claim 6, wherein the lactone is a macrocycle comprising at least 15 atoms in a ring and a substructure having the formula: 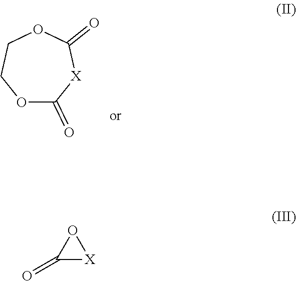
wherein
X is a saturated or unsaturated alkyl chain comprising 0-1 methyl groups.

19. The malodor-counteracting composition of claim 6, wherein the lactone is ethylene brassylate or (E)-oxacycloheptadec-10-en-2-one.

20. The malodor-counteracting composition of claim 4, wherein the one or more fragrance ingredients comprise phenethyl alcohol, tetrahydrolinalool, linalool, 3-phenylpropanal, cinnamaldehyde, decanal, ethyl linalool, dihydromyrcenol, 2-methyldecanal, (3E)-4-Methyl-3-decen-5-one, 4-Methyl-3-decen-5-ol (undecavertol), or any combination thereof.

21-22. (canceled)

23. The malodor-counteracting composition of claim 4, wherein (i) the 2,2′-(alkylimino)bis[ethanol]; (ii) the one or more fragrance ingredients; or (iii) both the 2,2′-(alkylimino)bis[ethanol] and the one or more fragrance ingredients are encapsulated in a capsule, with the proviso that both the 2,2′-(alkylimino)bis[ethanol] and the one or more fragrance ingredients are encapsulated in the same or separate capsules.

24. The malodor-counteracting composition of claim 23, wherein the capsule comprises a polymer selected from the group consisting of polyacrylate, polyurea, polyurethane, polyacrylamide, polyester, polyether, polyamide, poly(acrylate-co-acrylamide), starch, silica, gelatin and gum Arabic, alginate, chitosan, polylactide, poly(melamine-formaldehyde), poly(urea-formaldehyde) and any combination thereof.

25. (canceled)

26. The malodor-counteracting composition of claim 3, wherein the 2,2′-(alkylimino)bis[ethanol] is present in an amount ranging from about 60% wt to about 100% wt of the malodor-counteracting composition.

27. (canceled)

28. A consumer product comprising a malodor-counteracting composition of claim 3, wherein the consumer product is selected from the group consisting of a room freshener spray, a fragrance diffuser, a candle, a sachet, a clothes deodorant, a detergent, a fabric softener, a fabric refresher, a linen spray, a disposable diaper, a diaper pail deodorant, a baby wipe, an antiperspirant, a deodorant, a body wash, a soap, a shaving product, a hair care treatment, a hair care conditioner, a shampoo, an eye product, a baby cream, a men's face cream, a body lotion, a hair styling spray, a scent strip, a women's face cream, a facial makeup, a hand cream, a wipe or tissue, an intimate wipe, a leave-on insect repellent, a non-spray hair styling product, a body powder, a hand dishwashing product, a home cleaner, a toilet block, a garbage bag, a car freshener, a pet care product, and an animal litter material.

29. (canceled)

30. The consumer product of claim 28, wherein the 2,2′-(alkylimino)bis[ethanol] is present in the consumer product at a concentration of at least about 0.005% wt of the consumer product.

31. (canceled)

32. A method of counteracting a malodor in an air space or a substrate, comprising introducing into the air space or the substrate a malodor-counteracting composition according to claim 3, wherein the malodor is a body or environmental malodor selected from the group consisting of a sweat malodor, scalp malodor, a bathroom malodor, a mildew malodor, a mold malodor, a pet malodor, a smoke malodor and any combination thereof.

33-34. (canceled)

35. A malodor-counteracting composition comprising:
(a) a 2,2′-(alkylimino)bis[ethanol], wherein the alkyl is a C₄₋₁₂ alkyl; and
(b) one or more fragrance ingredients susceptible to hydrolysis, ethanolysis, solvolysis, retro-aldol elimination, dimerization, polymerization, and/or transesterification.
